(19) United States
(12) Patent Application Publication
(10) Pub. No.: US 2026/0308244 A1
(43) Pub. Date: Oct. 8, 2026

(54) TREATMENT OF AUTOIMMUNE DISEASES

(71) Applicant: UCB Developed Brands SRL, Brussels (BE)

(72) Inventors: Carlos Javier Cara Terribas, Brussels (BE); Yoshiya Tanaka, Fukuoka (JP); Bernard Robert Jean Lauwerys, Brussels (BE); Baran Ufuktepe, Brussels (BE); Ana Barbadillo Ruiz, Brussels (BE)

(21) Appl. No.: 19/483,448

(22) PCT Filed: May 10, 2024
(86) PCT No.: PCT/EP2024/062968

§ 371 Date: Nov. 11, 2025

(30) Foreign Application Priority Data
May 12, 2023 (EP) 23173178.7
Jun. 1, 2023 (EP) 23176822.7
Mar. 19, 2024 (EP) 24164645.4
Apr. 17, 2024 (EP) 24170908.8

(51) Int. Cl. C07K 16/24; A61K 31/42; A61K 31/519; A61K 39/00; A61P 37/06; G01N 33/68

(52) U.S. Cl. CPC C07K 16/241; A61K 31/42; A61K 31/519; A61P 37/06; G01N 33/6854; A61K 2039/505; A61K 2039/545; G01N 2333/46; G01N 2800/52

(57) ABSTRACT

The present disclosure provides methods of treating a rheumatic disease in difficult to treat human patients being seropositive for rheumatoid factor using a biologic disease modifying anti-rheumatic drug lacking an Fc fragment. In some embodiments, the method includes treating patients having rheumatoid arthritis being seropositive for rheumatoid factor using the certolizumab pegol.

### CROSS-REFERENCE TO RELATED APPLICATIONS

This application claims the right of priority based on European application serial no. 23173178.7, filed 12 May 2023, European application serial no. 23176822.7, filed 1 Jun. 2023, European application serial no. 24164645.4, filed 19 Mar. 2024, and European application serial no. 24170908.8, filed 17 Apr. 2024, which are herein incorporated in their entirety by reference.

### FIELD OF THE INVENTION

The present invention is in the field of treatment of autoimmune diseases, particularly rheumatoid arthritis, juvenile idiopathic arthritis, systemic lupus erythematosus, vasculitis and Sjogren's syndrome. The invention provides methods and compounds for the treatment of patients suffering from inflammatory autoimmune diseases, particularly rheumatoid arthritis.

### BACKGROUND OF THE INVENTION

Autoimmune diseases are a group of distinct disorders which share similar clinical, laboratory and immunological manifestations. Their fundamental pathobiological finding is the unfolding of an excessive self-reactive, antigen-driven, immune response characterized by inflammation and subsequent progressive tissue damage.

Rheumatic autoimmune diseases include conditions such as systemic lupus erythematosus (SLE), rheumatoid arthritis (RA), juvenile idiopathic arthritis, vasculitis and primary Sjögren's syndrome (pSS).

Rheumatoid arthritis (RA) is a chronic, symmetrical, inflammatory autoimmune disease that initially affects small joints, progressing to larger joints, and eventually the skin, eyes, heart, kidneys, and lungs. Often, the bone and cartilage of joints are destroyed, and tendons and ligaments weaken. All this damage to the joints causes deformities and bone erosion, usually very painful for a patient. Common symptoms of RA include morning stiffness of the affected joints for >30 min, fatigue, fever, weight loss, joints that are tender, swollen and warm, and rheumatoid nodules under the skin. The onset of this disease is usually from the age of 35 to 60 years, with remission and exacerbation.

There are three general classes of drugs commonly used in the treatment of rheumatoid arthritis: non-steroidal anti-inflammatory agents (NSAIDs), corticosteroids, and disease modifying anti-rheumatic drugs (DMARDs). NSAIDs and corticosteroids have a short onset of action while DMARDs can take several weeks or months to demonstrate a clinical effect. DMARDs include methotrexate, sulfasalazine, leflunomide, antimalarials as well as biological drugs such as etanercept, infliximab, adalimumab, certolizumab pegol, golimumab, abatacept, rituximab, tocilizumab, and anakinra. Other immunomodulators are occasionally used including azathioprine and cyclosporine. Because cartilage damage and bony erosions frequently occur within the first two years of disease, rheumatologists now move aggressively to a DMARD agent early in the course of disease, usually as soon as a diagnosis is confirmed.

Current treatment paradigm of RA is based on a ‘treat to target’ approach, i.e. treatments are added until the patient reaches a therapeutic target such as low disease activity (LDA). A ‘treat to target’ approach is associated with better long-term outcomes and decreased damage accrual. Biological DMARDs (bDMARDs), are effective in rapidly retarding the progression of the joint damage caused by RA. They are considered to be a more “direct, defined and targeted” method of treatment. Nonetheless, biologics pose the problem of serious side effects, such as increased risk of infections.

Among the bDMARDs etanercept, infliximab, adalimumab, golimumab, and certolizumab pegol are all TNFα inhibitors that prevent the recruitment of the cells that cause inflammation. Tumor necrosis factor α (TNFα) is a messenger protein that promotes inflammation in joints. Biological TNFα inhibitors are recommended if other second-line medications are not effective. Tumor necrosis factor alpha (TNFα) inhibitors have become a mainstay of treatment for rheumatoid arthritis (RA) patients after failed responses to conventional DMARDs. Despite the clinical efficacy of TNFα inhibitors (TNFi), many RA patients experience TNFi treatment failure due to the development of anti-drug antibodies (ADAs) that can reduce drug levels and lead to RA disease relapse (secondary failure). Furthermore, about 20-30% of RA patients treated with TNFα inhibitors exhibit primary failure (no efficacy response to at least 12-18 weeks of treatment).

60%-90% of patients with RA have detectable levels of rheumatoid factor (RF), a natural antibody which targets the fragment crystallizable (Fc) region of immunoglobulin (Ig) G, with high reactivity to the IgG1 isotype. Rheumatoid factors have been found as IgA, IgD, IgE, IgG and IgM isotypes and have been determined in pre-symptomatic individuals (Ingegnoli et al., Rheumatoid Factors: Clinical Applications, Dis Markers. 2013). Although IgM antibodies typically have a lower affinity for their antigens, little is known about the functional differences between these three isotypes. In its physiological role, pentameric IgM RF, the most prevalent RF subtype, increases the clearance of immune complexes by binding up to 10 IgG1-Fc regions per molecule of RF (FIG. 21). These large immune complexes are subsequently eliminated by endocytosis through binding to the cognate Fc receptor. There is evidence supporting the pathogenic role of RF with a key contribution to the pathophysiology of RA (Edwards et al., Rheumatoid arthritis: The predictable effect of small immune complexes in which antibody is also antigen, Br J Rheumatol. 1998). A high titer of RF in patients with RA has been associated with higher disease activity, disease progression and decreased response to treatment with TNFα inhibitors (Bobbio-Pallavicini F, et al., Ann Rheum Dis. 2007). Specifically, high RF titer≥50 U/mL has been significantly associated with RA disease activity compared with low titer or negative RF. Taken together, high titers RF are associated with more severe forms of RA that are poorly responsive to treatment with TNFα inhibitors.

Systemic lupus erythematosus (SLE) is a systemic autoimmune disease characterized by a loss if tolerance to chromatin and the production of antinuclear antibodies (e.g. anti-double stranded DNA, anti-Sm, anti-Ro, anti-La antibodies). Manifestations of the disease are pleiomorphic and include skin rashes, arthritis, fever, cytopenias, glomerulonephritis and central nervous system disorders. Anti-double stranded DNA antibodies are typically associated with more severe forms of the disease (i.e. lupus nephritis), while anti-Ro or anti-La antibodies are found preferentially in patients with non-nephritis symptoms, such as rashes or arthritis.

Rheumatoid factors (RF) are found in 20% patients with systemic lupus erythematosus (SLE), often in association with anti-Ro or anti-La antibodies and non-renal manifestations (Witte et al., Rheumatoid factors in systemic lupus erythematosus: association with clinical and laboratory parameters, Rheumatol Int 2000).

Treatment of SLE requires corticosteroids and other immunosuppressors such as azathioprine, mycophenolate mofetil, cyclophosphamide or calcineurin inhibitors. Biologics (e.g. rituximab, belimumab) are also increasingly used in patients with SLE and severe or refractory disease courses.

Primary Sjögren's syndrome (pSS) is a rare autoimmune disease characterized by polyclonal B cell activation, production of antinuclear antibodies, lymphocytic infiltration of lachrymal and salivary glands and a range of systemic symptoms, including arthritis, pancreatitis or interstitial nephropathy. Rheumatoid factors are found in about 50% of patients with pSS, often at high titers. Treatment options in pSS are limited and biologics are being developed to treat patients with severe manifestations of the disease.

Juvenile idiopathic arthritis is a group of rheumatic diseases which affect one or more joints for at least 6 weeks in a child at an age of 16 or younger. Arthritis, fever, rash, adenopathy, splenomegaly, and iridocyclitis are typical of some forms. Treatment involves intra-articular corticosteroids, nonsteroidal anti-inflammatory medicines (NSAIDs) and disease-modifying antirheumatic drugs (DMARDS), including biological DMARDS such as certain TNFα inhibitors.

There is thus a need for tailored therapeutic approaches for rheumatoid arthritis, juvenile idiopathic arthritis, SLE and Sjögren's syndrome patients with high RF titers.

### SUMMARY OF THE INVENTION

Whilst TNFα inhibitors have been used successfully for more than 20 years for the treatment of rheumatoid arthritis (RA), there are still subgroups of RA patients who do not respond well, or at all, initially to such treatment with a TNFα inhibitor or lose or fail to maintain the treatment effect over the period of treatment. A particular sub-group of patients for which this is a problem is RA patients who have a high titer of rheumatoid factor (RF). Typically, the titer of RF is measured in vitro in serum samples of patients.

This disclosure provides further analysis of data from 6 clinical studies: C-OPERA (NCT01451203), pooled RAPID trials (RAPID-1, [NCT00152386], RAPID-2 [NCT00160602], J-RAPID [NCT00791999], RAPID-C [NCT02151851]), and EXXELERATE (NCT01500278). Patients who received certolizumab pegol (CZP) or placebo/comparator adalimumab (ADA) with methotrexate (MTX) were categorized by baseline RF quartiles. Efficacy of CZP in patients with early and established RA across baseline RF levels was assessed with Disease Activity Score-28 erythrocyte sedimentation rate (DAS28-ESR). This post-hoc analysis of multiple clinical trials (phase 3 and 4) elucidates the efficacy of CZP across baseline RF quartiles in patients with early RA and established RA.

It has now been found, from this analysis of clinical data, that certolizumab pegol, a pegylated Fab′ fragment of an TNFα blocking antibody, displays the same level of efficacy in patients with RA with high RF titers as compared to low RF titers. This is contrary to other TNFα inhibitors such as adalimumab, infliximab, etanercept or golimumab that have lower efficacy in RA patients with high RF titers. Further, this analysis demonstrates that certolizumab pegol (CZP) is significantly more efficacious than adalimumab (ADA) in patients having a high titer of rheumatoid factor at baseline. Higher efficacy of certolizumab pegol in patients with high titers RF was associated with maintained plasma concentrations of the drug as compared to patients with low titers RF by opposition to adalimumab, the concentrations of which decreased over time in patients with high compared to low RF titers.

Accordingly, this disclosure describes methods of treating a rheumatic disorder in a human patient identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor at least 275 IU/ml.

The skilled artisan will understand that this finding will apply mutatis mutandis to other rheumatic diseases where patients have high RF titers, such as juvenile idiopathic arthritis, SLE and Sjögren's syndrome.

Accordingly, a first aspect of this disclosure is a method of treating a rheumatic disorder in a human patient seropositive for rheumatoid factor comprising administering a biologic drug lacking an Fc fragment to a human patient identified as having rheumatoid factor above 100 IU/ml, 121.4 IU/ml, 125 IU/ml, 145.9 IU/ml, 150 IU/ml, 175 IU/ml, 200 IU/ml or 204 IU/ml in serum, wherein (i) the therapeutic effect of the biologic drug lacking an Fc fragment is not reduced by RF in said patient and/or (ii) said treatment induces low disease activity or achieves substantial clinical benefit in said patient.

In some embodiments, this disclosure provides a method of treating rheumatoid arthritis in a human patient seropositive for rheumatoid factor comprising administering certolizumab pegol to a human patient identified as having rheumatoid factor above 100 IU/ml, 121.4 IU/ml, 125 IU/ml, 145.9 IU/ml, 150 IU/ml, 175 IU/ml, 200 IU/ml and more preferably 204 IU/ml in serum, wherein the (i) therapeutic effect of certolizumab pegol is not reduced by RF in said patient and/or (ii) the treatment induces low disease activity or achieves substantial clinical benefit in said patient.

In some embodiments, this disclosure provides a method of treating rheumatoid arthritis in a human patient seropositive for rheumatoid factor comprising (a) determining the titer of rheumatoid factor in a sample, preferably a serum sample, from said patient, and (b) administering certolizumab pegol to said patient if the titer of rheumatoid factor is above 100 IU/ml, 121.4 IU/ml, 125 IU/ml, 145.9 IU/ml, 150 IU/ml, 175 IU/ml, 200 IU/ml and more preferably 204 IU/ml in serum, wherein the (i) therapeutic effect of certolizumab pegol is not reduced by RF in said patient and/or (ii) the treatment induces low disease activity or achieves substantial clinical benefit in said patient.

In some embodiments, this disclosure provides a method of maintaining low disease activity or remission of a rheumatic disease in a human subject in need thereof, where the method includes administering to the subject a dose of a bDMARD lacking an Fc fragment that is effective to maintain low disease activity or remission in the subject, wherein the subject is identified as having an elevated baseline rheumatoid factor (RF) serum level of more than 100 IU/ml.

### BRIEF DESCRIPTION OF THE FIGURES

FIG. 1 shows data from a prospective trial comparing certolizumab pegol to a TNFi with an Fc fragment (adalimumab), both in association with methotrexate, in patients with moderate to severe rheumatoid arthritis. Impact of both drugs on disease activity (DAS28-CRP in the upper panel, proportion of patients reaching LDA in the lower panel) is identical in the entire population of patients.

FIG. 2 shows that certolizumab pegol exhibits a different pattern of response to therapy compared to a TNFi with an Fc fragment (adalimumab) when patients are categorized according to RF titers. In patients with high titers RF, response to a TNFi with a Fc fragment (adalimumab) is decreased compared to patients with low RF titers, resulting in higher DAS28-CRP values (upper panel). By contrast, effect of certolizumab pegol is not influenced by RF titers (lower panel) and DAS28-CRP values are interwoven.

FIG. 3A shows that certolizumab pegol and a TNFi with an Fc fragment (adalimumab) display similar clinical efficacy (DAS28-CRP scores) in patients with low RF titers (FIG. 3a upper panel). By contrast, in patients with high RF titers, administration of certolizumab pegol results in lower DAS28-CRP scores (lower panel).

FIG. 3B shows response to CZP and ADA to Week 104 measured by (panel A) DAS28-CRP and (panel B) DAS28-CRP LDA, stratified by RF quartile (RF≤204 UI/mL or RF>204 UI/mL) at baseline [OC; NRI]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). N are for OC data. [a] Defined as DAS28-CRP≤2.7. [b] N at Week 2.ADA: adalimumab; CRP: C-reactive protein; CZP: certolizumab pegol; DAS28: Disease Activity Score-28 joint count; LDA: low disease activity; MTX: methotrexate: NRI: non-responder imputation; OC: observed case; Q3: third quartile; RF: rheumatoid factor; SD: standard deviation; TNFi: tumor necrosis factor inhibitor.

FIG. 4 shows that in RA patients with high RF titers, a lower proportion of patients treated with a TNFi with a Fc fragment (adalimumab) reach LDA, compared to RA patients with low RF titers. This pattern is not observed with certolizumab pegol. Similar proportions of patients treated with certolizumab pegol reach LDA whether they have low or high TF titers.

FIG. 5A shows how high RF titers influence plasma drug concentrations of a TNFi with an Fc fragment (adalimumab) but not certolizumab pegol. Drug concentrations of certolizumab pegol (left panels) and adalimumab (right panels) are displayed in function of RF titers. High RF titers are associated with lower drug concentrations of adalimumab compared to low RF titers. By contrast, drug concentrations of certolizumab pegol are not different in high versus low RF titers.

FIG. 5B shows mean drug plasma concentrations of (panel A) CZP and (panel B) ADA to Week 104, stratified by RF quartile (RF≤204 UI/mL or RF>204 UI/mL) at baseline [OC]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). ADA: adalimumab; CZP: certolizumab pegol; MTX: methotrexate; OC: observed case; Q3: third quartile; RF: rheumatoid factor; SD: standard deviation.

FIG. 6 shows the study designs of the clinical trials RAPID-1, RAPID-2, J-RAPID, RAPID-C, C-OPERA, and EXXELERATE. ACR20: ≥20% improvement based on American College of Rheumatology criteria; ADA: adalimumab; CZP: certolizumab pegol; DAS28 (ESR): Disease Activity Score 28-erythrocyte sedimentation rate; MTX: methotrexate; OLE: open-label extension; PBO: placebo; Q2W: every two weeks; Q4W: every four weeks; RCT: randomized-controlled trial; Wk: week.

FIG. 7, panels (a)-(c) show the DAS28 (ESR) LDA and REM rates at Weeks 12 and 24 for the (panel a) C-OPERA, (panel b) Pooled RAPID, and (panel c) EXXELERATE studies. RF quartiles for Week 24 were defined as ≤32.0 IU/mL, >32.0-≤74.0 IU/mL, >74.0-≤204.0 IU/mL, and >204 IU/mL for Q1-4, respectively. DAS28 (ESR) 2.6-≤3.2 was classified as LDA and DAS28 (ESR)<2.6 was classified as REM. ADA: adalimumab; CZP: certolizumab pegol; DAS28 (ESR): Disease Activity Score 28-erythrocyte sedimentation rate; LDA: low disease activity; MTX: methotrexate; PBO: placebo; Q: quartile; REM: remission; RF: rheumatoid factor.

FIGS. 8A-8C show the mean DAS28 (ESR) over 24 weeks for the C-OPERA (FIG. 8A), Pooled RAPID (FIG. 8A), and EXXELERATE (FIG. 8C) studies. Week 24 data are mean (SD). ADA: adalimumab; CZP: certolizumab pegol; DAS28 (ESR): Disease Activity Score 28-erythrocyte sedimentation rate; MTX: methotrexate; PBO: placebo; Q: quartile; RF: rheumatoid factor; SD: standard deviation.

FIG. 8D shows response to CZP and ADA to Week 104 measured by (panel A) DAS28-ESR and (panel B) DAS28-ESR LDA, stratified by RF quartile (RF≤204 UI/mL or RF>204 UI/mL) at baseline [OC; NRI]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). N are for OC data. [a] Defined as DAS28-ESR≤3.2. [b] N at Week 2. ADA: adalimumab; CZP: certolizumab pegol; DAS28: Disease Activity Score-28 joint count; ESR: erythrocyte sedimentation rate; LDA: low disease activity; MTX: methotrexate; NRI: non-responder imputation; OC: observed case; Q3: third quartile; RF: rheumatoid factor; SD: standard deviation; TNFi: tumor necrosis factor inhibitor.

FIG. 9 shows that in an in vitro ELISA experiment an anti-IgG Fc antibody bound significantly to 7 different biological drugs that are used for the treatment of RA and other rheumatic diseases but it did not bind to certolizumab pegol. In the ELISA plastic plates were coated with 8 different biologics drugs: abatacept (ABT), tocilizumab (TCZ), sarilumab (SAR), etanercept (ETN), infliximab (IFX), adalimumab (ADA), golimumab (GLM) and certolizumab pegol (CZP). Then, the following secondary antibodies conjugated with alkaline phosphatase were added to the above-mentioned coated ELISA plates in separate experiments: (1) antibody against IgG Fc, (2) antibody against IgM Fc, (3) antibody against the κ type antibody light chain and (4) antibody against the λtype antibody light chain.

FIG. 10 shows that in the ELISA experiment described in FIG. 9 above the anti-IgM Fc antibody did not bind to the 8 biological drugs, including not to certolizumab pegol. This confirmed that the binding of anti-IgG Fc antibody mentioned in FIG. 9 is specific.

FIG. 11 shows that in the ELISA experiment described in FIG. 9 above the anti-κ type antibody light chain antibody bound to TCZ, SAR, IFX, ADA, GLM and CZP all of which are known to have a κ type antibody light chain. ABT and ETN were not bound. ABT and ETN are fusion proteins which do not have a light chain. This confirmed that the binding of anti-IgG Fc antibody mentioned in FIG. 9 is specific.

FIG. 12 shows data resulting from an experiment similar to the one of FIG. 13 with a focus on two positive and negative sera. Optical density is higher in RF positive cases across all the bDMARDs except CZP. In the two cases using RF negative sera, OD is zero because no RF can bind to the Fc region of the bDMARDs (all) or no Fc region is present in the drug in the case of CZP.

FIG. 13 shows that in an in vitro ELISA experiment, 7 different biological drugs that are used for the treatment of RA and other rheumatic diseases [abatacept (ABT), tocilizumab (TCZ), sarilumab (SAR), etanercept (ETN), infliximab (IFX), adalimumab (ADA) and golimumab (GLM)], but not certolizumab pegol (CZP), formed immune complexes when brought in contact with diluted serum containing RF. As a control, it is also shown that none of the 7 drugs and also not certolizumab pegol formed immune complexes when brought in contact with diluted serum which did not contain RF. It also shows the occurrence and size of immune-complexes made of RF different bDMARDs with an Fc region and without an Fc region (only CZP). Both aspects are measured by mean of OD (Optical Density, the higher optical density meaning more and/or larger immune-complexes). All bDMARDs exhibit a high OD in the experiment (indicting that RF binds to the Fc region), when exposed to positive RF sera. No binding is observed when serum is RF negative. Only CZP does not show any type of binding (OD=zero) when exposed to RF positive or negative serum.

FIG. 14 shows response to CZP and ADA to Week 104 measured by (panel A) CDAI and (panel B) CDAI LDA, stratified by RF quartile (RF≤204 UI/mL or RF>204 UI/mL) at baseline [OC]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to. [a] Defined as CDAI≤10. [b] N at Week 2. ADA: adalimumab; CDAI: Clinical Disease Activity Index; CZP: certolizumab pegol; MTX: methotrexate: OC: observed case; Q3: third quartile; RF: rheumatoid factor; SD: standard deviation; TNFi: tumor necrosis factor inhibitor.

FIG. 15 shows response to CZP and ADA to Week 104 measured by (panel A) SDAI and (panel B) SDAI LDA, stratified by RF quartile (RF≤204 UI/mL or RF>204 UI/mL) at baseline [OC]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). A high SDAI score indicates increased disease severity. [a] Defined as SDAI≤11]. [b] N at Week 2. ADA: adalimumab; CDAI: Clinical Disease Activity Index; CZP: certolizumab pegol; MTX: methotrexate: OC: observed case; Q3: third quartile; RF: rheumatoid factor; SD: standard deviation; SDAI: Simple Disease Activity Index; TNFi: tumor necrosis factor inhibitor.

FIG. 16 shows HAQ-DI score in CZP and ADA treated patients to Week 104, stratified by RF quartile (RF≤204 UI/mL or RF>204 UI/mL) at baseline [OC]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). ADA: adalimumab; CZP: certolizumab pegol; MTX: methotrexate: HAQ-DI: Health Assessment Questionnaire-Disability Index; OC: observed case; Q3: third quartile; RF: rheumatoid factor; SD: standard deviation; TNFi: tumor necrosis factor inhibitor.

FIG. 17 shows response to CZP and ADA to Week 104 measured by the proportion of patients achieving Boolean remission, stratified by RF quartile (RF≤204 UI/mL or RF>204 UI/mL) at baseline [NRI]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). Boolean remission is defined as SJC≤1 and TJC≤1 and PtGADA≤2 and CRP≤1. ADA: adalimumab; CRP: C-reactive protein; CZP: certolizumab pegol; MTX: methotrexate; NRI: non-responder imputation; PtGADA: Patient's Global Assessment of Disease Activity; Q3: third quartile; RF: rheumatoid factor; SJC: swollen joint count; TJC: tender joint count; TNFi: tumor necrosis factor inhibitor.

FIG. 18 shows mean DAS28-CRP (panel A) and DAS28-ESR (panel B) in CZP and ADA treated patients after exclusion of patients with high levels of anti-drug antibodies (>50th percentile), stratified by RF quartile (RF≤204 UI/mL or RF>204 UI/mL), to Week 104 [OC]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). ADA: adalimumab; CRP: C-reactive protein; CZP: certolizumab pegol; DAS28: Disease Activity Score-28 joint count; ESR: erythrocyte sedimentation rate; MTX: methotrexate: OC: observed case; Q3: third quartile; RF: rheumatoid factor; SD: standard deviation; TNFi: tumor necrosis factor inhibitor.

FIG. 19 shows mean DAS28-CRP LDA (panel A) and DAS28-ESR LDA (panel B) in CZP and ADA treated patients after exclusion of patients with high levels of anti-drug antibodies (>50th percentile), stratified by RF quartile (RF≤204 UI/mL or RF>204 UI/mL), to Week 104 [OC]. Quartiles indicate RF level at baseline. Full analysis set. Data reported according to the treatment patients were on at time of measurement (i.e., any patients who had switched TNFi at Week 12 were subsequently included in the arm for their new treatment, rather than the arm they were initially randomized to). [a] Defined as DAS28-CRP≤2.7. [b] N at Week 2. [c] Defined as DAS28-ESR≤3.2. ADA: adalimumab; CRP: c-reactive protein; CZP: certolizumab pegol; DAS28: Disease Activity Score-28 joint count; ESR: erythrocyte sedimentation rate; LDA: low disease activity; MTX: methotrexate; OC: observed case; Q3: third quartile; RF: rheumatoid factor; TNFi: tumor necrosis factor inhibitor.

FIG. 20 shows response to CZP and ADA to Week 104 measured by (panel A) DAS28-CRP and (panel B) DAS28-ESR, stratified by ACPA level at baseline [OC]. Full Analysis Set to Week 12; Week 12 Full Analysis Set from Week 18. Data reported according to the treatment patients were on at time of measurement (i.e., any patients who had switched TNFi at Week 12 were subsequently included in the arm for their new treatment, rather than the arm they were initially randomized to). ACPA: anti-citrullinated protein antibody; ADA: adalimumab; CRP: c-reactive protein; CZP: certolizumab pegol; DAS28: Disease Activity Score-28 joint count; ESR: erythrocyte sedimentation rate; MTX: methotrexate: OC: observed case; Q3: third quartile; SD: standard deviation; TNFi: tumor necrosis factor inhibitor.

FIG. 21 illustrates how rheumatoid factors of the IgM type bind to Fc region of IgG in general, including to therapeutic antibodies such as TNF inhibitors to form large immune complexes. These immune complexes are internalized by macrophages through the Fc gamma receptor and degraded by the lysosome.

FIG. 22 illustrates the molecular structure of bDMARDs most of which have an Fc region. An example of a bDMARD without an Fc region is certolizumab pegol.

FIG. 23 shows the set up of an IgM-RF binding ELISA assay. bDMARD is coated to a microtiter plate and then IgM RF from serum is added to the plate. Thereafter binding of IgM RF is detected with anti-human IgM antibody.

FIG. 24 shows the 4 quartiles of RF concentrations in the population post PS-ITPW from FIRST Registry.

FIG. 25 shows graphs of the CDAI at weeks 0, 2 and 4 in patients from FIRST Registry post PS-ITPW treated with adalimumab (ADA) or CZP. There is a significantly lower disease activity in patients with baseline RF above 121.4 treated with CZP (p=0.034).

FIG. 26 shows the CDAI remission rate in patients post PS-ITPW treated with adalimumab (ADA) or CZP. The subjects in Q4 (RF>121.4 IU/ml) show significantly higher CDAI remission rate (p=0.008) when treated with CZP vs ADA.

FIG. 27 shows the baseline RF quartiles.

FIG. 28 shows the CDAI remission rate comparison Q1-Q3 vs Q4 across different anti-TNF drugs (bDMARDs).

### DETAILED DESCRIPTION OF THE INVENTION

It has now surprisingly been found by the present inventors that a TNFα inhibitor lacking an Fc fragment (certolizumab pegol) exhibits a different pattern of response to therapy as compared to a TNFα inhibitor having an Fc fragment (adalimumab) in patients with high versus low titers of RF. We observed differences in patterns of plasma drug concentrations between both bDMARDs. Thus, mean adalimumab drug concentrations were lower in patients with high compared to low titers of RF in the patient. By contrast, this difference was not observed with certolizumab pegol that maintained similar drug concentrations in patients with high compared to low RF titers.

Taken together, our observations indicate that certolizumab pegol does not behave as expected in patients with high titers RF. By opposition to adalimumab, certolizumab pegol maintains therapeutic responses and plasma drug concentrations in patients with high titers RF, similar to these observed in patients with low titers RF.

This disclosure provides methods of treating a rheumatic disorder in a human patient identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

Accordingly, in a first embodiment this disclosure provides a method of treating rheumatoid arthritis in a human patient seropositive for rheumatoid factor comprising administering a bDMARD lacking an Fc fragment, preferably certolizumab pegol, to a human patient identified as having rheumatoid factor above 100 IU/ml, 121.4 IU/ml, 125 IU/ml, 145.9 IU/ml, 150 IU/ml, 175 IU/ml, 200 IU/ml and more preferably 204 IU/ml in serum, wherein (i) the therapeutic effect of the bDMARD lacking an Fc fragment, preferably certolizumab pegol is not reduced by RF in said patient and/or (ii) the treatment induces low disease activity or achieves substantial clinical benefit in said patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

A second embodiment of this disclosure is a method of treating rheumatoid arthritis in a human patient seropositive for rheumatoid factor comprising (a) determining the titer of rheumatoid factor in said patient, and (b) administering a bDMARD lacking an Fc fragment, preferably certolizumab pegol, to said patient if the titer of rheumatoid factor is above 100 IU/ml, 125 IU/ml, 150 IU/ml, 175 IU/ml or 200 IU/ml and more preferably 204 IU/ml in serum, wherein (i) the therapeutic effect of the bDMARD lacking an Fc fragment, preferably certolizumab pegol, is not reduced by RF in said patient and/or (i) the treatment induces low disease activity or achieves substantial clinical benefit in the patient. In some embodiments, the titer of rheumatoid factor in said patient is above 100 IU/ml. In some embodiments, the titer of rheumatoid factor in said patient is above 121.4 IU/ml. In some embodiments, the titer of rheumatoid factor in said patient is above 125 IU/ml. In some embodiments, the titer of rheumatoid factor in said patient is above 145.9 IU/ml. In some embodiments, the titer of rheumatoid factor in said patient is above 150 IU/ml. In some embodiments, the titer of rheumatoid factor in said patient is above 175 IU/ml. In some embodiments, the titer of rheumatoid factor in said patient is above 200 IU/ml. In some embodiments, the titer of rheumatoid factor in said patient is above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

It will be understood by the skilled artisan that the surprising finding that the activity of a biological DMARD (bDMARD) lacking an Fc fragment such as certolizumab pegol is not reduced in a patient having a high titer of RF can be applied mutatis mutandis to other rheumatic diseases and other bDMARDs lacking an Fc fragment. Such bDMARD lacking an Fc fragment, e.g. certolizumab pegol, dapirolizumab pegol or ozoralizumab, would thus be a preferable drug for the treatment of patients having a rheumatic disease and high RF titers, such as e.g. juvenile idiopathic arthritis, vasculitis, SLE and Sjögren's syndrome.

According, in a third embodiment this disclosure provides a method of treating juvenile idiopathic arthritis in a human patient seropositive for rheumatoid factor comprising administering a bDMARD lacking an Fc fragment, preferably dapirolizumab pegol or ozoralizumab and most preferably certolizumab pegol, to a human patient identified as having rheumatoid factor above 100 IU/ml, 121.4 IU/ml, 125 IU/ml, 145.9 IU/ml, 150 IU/ml, 175 IU/ml, or 200 IU/ml and more preferably above 204 IU/ml in serum, wherein (i) the therapeutic effect of the bDMARD lacking an Fc fragment, preferably dapirolizumab pegol or ozoralizumab and most preferably certolizumab pegol, is not reduced by RF in said patient and/or (ii) the treatment induces low disease activity or achieves substantial clinical benefit in said patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor at least 275 IU/ml.

A fourth embodiment of this disclosure is a method of treating juvenile idiopathic arthritis in a human patient seropositive for rheumatoid factor comprising (a) determining the titer of rheumatoid factor in said patient, and (b) administering a bDMARD lacking an Fc fragment, preferably dapirolizumab pegol or ozoralizumab and most preferably certolizumab pegol, to said patient if the titer of rheumatoid factor is above 100 IU/ml, 121.4 IU/ml, 125 IU/ml, 145.9 IU/ml, 150 IU/ml, 175 IU/ml, 200 IU/ml, and more preferably 204 IU/ml in serum, wherein (i) the therapeutic effect of the bDMARD lacking an Fc fragment, preferably dapirolizumab pegol or ozoralizumab and most preferably certolizumab pegol, is not reduced by RF in said patient and/or (i) the treatment induces low disease activity or achieves substantial clinical benefit in the patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

In a fifth embodiment this disclosure provides a method of treating systemic lupus erythematosus (SLE) in a human patient seropositive for rheumatoid factor comprising administering a bDMARD lacking an Fc fragment, preferably dapirolizumab pegol, to a human patient identified as having rheumatoid factor above 100 IU/ml, 121.4 IU/ml, 125 IU/ml, 145.9 IU/ml, 150 IU/ml, 175 IU/ml, 200 IU/ml and more preferably 204 IU/ml in serum, wherein (i) the therapeutic effect of the bDMARD lacking an Fc fragment, preferably dapirolizumab pegol, is not reduced by RF in said patient and/or (ii) the treatment induces low disease activity or achieves substantial clinical benefit in said patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

A sixth embodiment of this disclosure is a method of treating systemic lupus erythematosus (SLE) in a human patient seropositive for rheumatoid factor comprising (a) determining the titer of rheumatoid factor in said patient, and (b) administering a bDMARD lacking an Fc fragment, preferably dapirolizumab pegol, to said patient if the titer of rheumatoid factor is above 100 IU/ml, 125 IU/ml, 150 IU/ml, 175 IU/ml or 200 IU/ml in serum and more preferably 204 IU/ml in serum, wherein (i) the therapeutic effect of the bDMARD lacking an Fc fragment, preferably dapirolizumab pegol, is not reduced by RF in said patient and/or (i) the treatment induces low disease activity or achieves substantial clinical benefit in the patient. In some embodiments, the titer of rheumatoid factor in said patient is above 100 IU/ml. In some embodiments, the titer of rheumatoid factor in said patient is above 121.4 IU/ml. In some embodiments, the titer of rheumatoid factor in said patient is above 125 IU/ml. In some embodiments, the titer of rheumatoid factor in said patient is above 145.9 IU/ml. In some embodiments, the titer of rheumatoid factor in said patient is above 150 IU/ml. In some embodiments, the titer of rheumatoid factor in said patient is above 175 IU/ml. In some embodiments, the titer of rheumatoid factor in said patient is above 200 IU/ml. In some embodiments, the titer of rheumatoid factor in said patient is above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

In a seventh embodiment this disclosure provides a method of treating Sjögren's syndrome, preferably primary Sjögren's syndrome (pSS), in a human patient seropositive for rheumatoid factor comprising administering a bDMARD lacking an Fc fragment, preferably certolizumab pegol or ozoralizumab and most preferably dapirolizumab pegol, to a human patient identified as having rheumatoid factor above 100 IU/ml, 121.4 IU/ml, 125 IU/ml, 145.9 IU/ml, 150 IU/ml, 175 IU/ml, 200 IU/ml or 200 IU/ml and more preferably 204 IU/ml in serum, wherein (i) the therapeutic effect of the bDMARD lacking an Fc fragment, preferably certolizumab pegol or ozoralizumab and most preferably dapirolizumab pegol, is not reduced by RF in said patient and/or (ii) the treatment induces low disease activity or achieves substantial clinical benefit in said patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

An eighth embodiment of this disclosure is a method of treating Sjögren's syndrome, preferably primary Sjögren's syndrome (pSS), in a human patient seropositive for rheumatoid factor comprising (a) determining the titer of rheumatoid factor in said patient, and (b) administering a bDMARD lacking an Fc fragment, preferably certolizumab pegol or ozoralizumab and most preferably dapirolizumab pegol, to said patient if the titer of rheumatoid factor is above 100 IU/ml, 121.4 IU/ml, 125 IU/ml, 145.9 IU/ml, 150 IU/ml, 175 IU/ml, 200 IU/ml and more preferably 204 IU/ml in serum, wherein (i) the therapeutic effect of the bDMARD lacking an Fc fragment, preferably certolizumab pegol or ozoralizumab and most preferably dapirolizumab pegol, is not reduced by RF in said patient and/or (i) the treatment induces low disease activity or achieves substantial clinical benefit in the patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

In a further embodiment this disclosure provides a method of treating vasculitis in a human patient seropositive for rheumatoid factor comprising administering a bDMARD lacking an Fc fragment, preferably dapirolizumab pegol or ozoralizumab and most preferably certolizumab pegol, to a human patient identified as having rheumatoid factor above 100 IU/ml, 121.4 IU/ml, 125 IU/ml, 145.9 IU/ml, 150 IU/ml, 175 IU/ml, or 200 IU/ml and more preferably above 204 IU/ml in serum, wherein (i) the therapeutic effect of the bDMARD lacking an Fc fragment, preferably dapirolizumab pegol or ozoralizumab and most preferably certolizumab pegol, is not reduced by RF in said patient and/or (ii) the treatment induces low disease activity or achieves substantial clinical benefit in said patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

In a further embodiment of this disclosure is a method of treating vassculitis in a human patient seropositive for rheumatoid factor comprising (a) determining the titer of rheumatoid factor in said patient, and (b) administering a bDMARD lacking an Fc fragment, preferably dapirolizumab pegol or ozoralizumab and most preferably certolizumab pegol, to said patient if the titer of rheumatoid factor is above 100 IU/ml, 121.4 IU/ml, 125 IU/ml, 145.9 IU/ml, 150 IU/ml, 175 IU/ml, 200 IU/ml, and more preferably 204 IU/ml in serum, wherein (i) the therapeutic effect of the bDMARD lacking an Fc fragment, preferably dapirolizumab pegol or ozoralizumab and most preferably certolizumab pegol, is not reduced by RF in said patient and/or (i) the treatment induces low disease activity or achieves substantial clinical benefit in the patient. In some embodiments, the human patient is identified as having rheumatoid factor above 100 IU/ml. In some embodiments, the human patient is identified as having rheumatoid factor above 121.4 IU/ml. In some embodiments, the human patient is identified as having rheumatoid factor above 125 IU/ml. In some embodiments, the human patient is identified as having rheumatoid factor above 145.9 IU/ml. In some embodiments, the human patient is identified as having rheumatoid factor above 150 IU/ml. In some embodiments, the human patient is identified as having rheumatoid factor above 175 IU/ml. In some embodiments, the human patient is identified as having rheumatoid factor above 200 IU/ml. In some embodiments, the human patient is identified as having rheumatoid factor above 204 IU/ml. In some embodiments, the human patient is identified as having rheumatoid factor above 225 IU/ml. In some embodiments, the human patient is identified as having rheumatoid factor above 250 IU/ml. In some embodiments, the human patient is identified as having rheumatoid factor at least 275 IU/ml.

A ninth embodiment of this disclosure is the method of treating according to any one of the first, second, third, fourth, fifth or sixth embodiment of the invention wherein the low disease activity is achieved after 90 days, 100 days, 104 days, 120 days or 180 days after the first administration of the bDMARD lacking an Fc fragment.

A tenth embodiment of this disclosure is the method of treating according to any one of the first, second, third, fourth, fifth, sixth, seventh, eighth or nineth embodiment of the invention wherein the low disease activity or the substantial clinical benefit in the patient is achieved with a likelihood of 40%, 45%, 50% or 55%.

An eleventh embodiment of this disclosure is the method of treating according to any one of the first, second, third, fourth, nineth or tenth embodiment of the invention wherein certolizumab pegol is administered to the patient in step (b) at loading dose of 400 mg initially and 400 mg two weeks later followed by a maintenance dose of (i) 400 mg of every 4 weeks or (ii) 200 mg every 2 weeks.

A twelfth embodiment of this disclosure is the method of treating according to any one of the first, second, third, fourth, fifth, sixth, seventh, eighth, nineth, tenth or eleventh embodiment of the invention wherein no loading dose is administered to the patient at the initiation of treatment.

A thirteenth embodiment of this disclosure is the method of treating according to any one of any one of the first, second, third, fourth, fifth, sixth, seventh, eighth, nineth, tenth, eleventh or twelfth embodiment of the invention wherein the rheumatoid factor is of the IgM, IgG, IgD, IgE or IgA isotype.

A fourteenth embodiment of this disclosure is the method of treating according to any one of the first, second, third, fourth, fifth, sixth, seventh, eighth, nineth, tenth, eleventh, twelfth or thirteenth embodiment of the invention wherein the bDMARD, preferably certolizumab pegol in case of the treatment of RA or juvenile idiopathic arthritis and dapirolizumab in case of the treatment of SLE, is administered in combination with another anti-rheumatic drug, preferably wherein the anti-rheumatic drug is non-biological DMARD, such as e.g. methotrexate or leflunomide, or a NSAID.

A fifteenth embodiment of this disclosure is the method of treating according to any one of the first, second, third, fourth, fifth, sixth, seventh, eighth, ninth, tenth, eleventh, twelfth, thirteenth or fourteenth embodiment of the invention wherein the patient has failed to respond to treatment with a biological DMARD (bDMARD) having an Fc fragment, e.g a TNFα inhibitor, such as adalimumab, infliximab, etanercept, or golimumab; a T cell costimulatory inhibitor, such as abatacept; an IL-6 or IL-6 receptor inhibitor, such as tocilizumab, sarilumab or olokizumab, or an anti-CD20 antibody, such as rituximab; or anakinra; or belimumab.

A sixteenth embodiment of this disclosure is the method of treating according to the fifteenth embodiment of this disclosure wherein treatment failure is a primary failure.

A seventeenth embodiment of this disclosure is the method of treating according to the fifteenth embodiment of the invention wherein treatment failure is a secondary failure.

An eighteenth embodiment of this disclosure is the method of treating according to any one of the first, second, third, fourth, fifth, sixth, seventh, eighth, ninth, tenth, eleventh, twelfth, thirteenth, fourteenth, fifteenth, sixteenth or seventeenth embodiment of the invention wherein the patient has failed to respond to treatment with a non-biological DMARD.

A nineteenth embodiment of this disclosure is the method of treating according to the eighteenth embodiment of the invention wherein the DMARD is methotrexate, leflunomide, sulfasalazine, chloroquine, hydroxychloroquine, gold, azathioprine, ciclosporin, mycofenolate mofetil or cyclophosphamide.

A twentieth embodiment of this disclosure is the method of treating according to any one of the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment of the invention wherein the bDMARD lacking an Fc fragment, preferably certolizumab pegol in the case of treatment of a patient with RA or juvenile arthritis and dapirolizumab pegol in the case of treatment of a patient with SLE, is administered chronically, for example at regular intervals, to a patient identified as having rheumatoid factor above 100 IU/ml, 121.4 IU/ml, 125 IU/ml, 145.9 IU/ml, 150 IU/ml, 175 IU/ml, 200 IU/ml or 204 IU/ml in serum, for example at regular intervals, for at least for 3 months, 6 months, 9 months, 12 months, 15 months, 18 months, 2 years or more than 2 years.

A twenty-first embodiment of this disclosure is the method of treating according to any one of the first, second, third, fourth, fifth, sixth, seventh, eighth, ninth, tenth, eleventh, twelfth, thirteenth, fourteenth, fifteenth, sixteenth, seventeenth, eighteenth, nineteenth or twentieth embodiment of the invention wherein the RF in the patient does not decrease the concentration of the bDMARD lacking an Fc fragment, preferably certolizumab pegol in the case of treatment of a patient with RA or juvenile arthritis and dapirolizumab pegol in the case of treatment of a patient with of SLE, following administration.

The titer of rheumatoid factor in patients suffering from RA, juvenile arthritis, SLE, Sjögren's syndrome or other rheumatic diseases can be determined for example by large scale automated methods involving for example nephelometric and turbidimetric techniques. These methods are not isotype-nonspecific, although they mainly detected IgM RF. Isotype-specific ELISAs and other enzyme immunoassays can be used to detect and quantify RFs in patients overcoming this hurdle. Commercial addressable laser bead immunoassays (ALBIA) can also be used for the measurement of RF titer in patients (Rönnelid et al., Autoantibodies in Rheumatoid Arthritis-Laboratory and Clinical Perspectives, Front. Immunol., 2021).

The methods of this disclosure can be directed to treatment of rheumatic disorders or diseases in human subjects in need thereof, including diseases where patients have high RF serum levels. In some embodiments of the methods of this disclosure, the human subject has rheumatoid arthritis (RA). In some embodiments of the methods of this disclosure, the human subject has a rheumatic disease, such as juvenile idiopathic arthritis, SLE or Sjögren's syndrome.

In some embodiments of the methods of this disclosure, the human subject has a high or elevated RF serum level. In some embodiments, the human subject has a high titer of RF, as measured in vitro from a serum sample. The rheumatoid factor that is measured can be of the IgM, IgG, IgD, IgE or IgA isotype.

In some embodiments, the subject has, or is identified as having, a RF serum level (or titer) above 100 IU/ml. In some embodiments, the subject has RF serum level above 121.4 IU/ml. In some embodiments, the subject has RF serum level above 125 IU/ml. In some embodiments, the subject has RF serum level above 145.9 IU/ml. In some embodiments, the subject has RF serum level above 150 IU/ml. In some embodiments, the subject has RF serum level above 175 IU/ml. In some embodiments, the subject has RF serum level above 200 IU/ml. In some embodiments, the subject has RF serum level above 204 IU/ml. In some embodiments, the subject has RF serum level above 225 IU/ml. In some embodiments, the subject has RF serum level above 250 IU/ml. In some embodiments, the subject has RF serum level of at least 275 IU/ml.

The methods of this disclosure provide for maintenance of plasma drug levels of the bDMARD agents that are administered to the subject, even when the subject has particular elevated RF levels. In some embodiments of the methods of this disclosure, the method includes administering a bDMARD lacking an Fc fragment. In some embodiments, the bDMARD lacking an Fc fragment is certolizumab pegol. It is understood that any convenient salt forms of the bDMARD are encompassed by the names of the agents described herein. In some embodiments, the bDMARD is dapirolizumab pegol.

In some embodiments of the methods of this disclosure, the human patient maintains low disease activity or remission of the rheumatic disease for 90 days, 100 days, 104 days, 120 days or 180 days after the first administration of the bDMARD lacking an Fc fragment.

In some embodiments of the methods of this disclosure, certolizumab pegol is administered to the patient in step (b) at loading dose of 400 mg initially and 400 mg two weeks later followed by a maintenance dose. In some embodiments of the methods of this disclosure, no loading dose is administered to the patient at the initiation of treatment.

In some embodiments of the methods of this disclosure, certolizumab pegol is administered at a dose of 400 mg. In some embodiments of the methods of this disclosure, certolizumab pegol is administered at a dose of 200 mg. In some embodiments of the methods of this disclosure, certolizumab pegol is administered subcutaneously.

In some embodiments of the methods of this disclosure, certolizumab pegol is administered at a dose of 400 mg of every 4 weeks. In some embodiments of the methods of this disclosure, certolizumab pegol is administered at a dose of 200 mg every 2 weeks.

In some embodiments of the methods of this disclosure, the bDMARD is administered chronically, for example at regular intervals, to a patient identified as having an elevated rheumatoid factor, for example at regular intervals, for at least for 3 months, 6 months, 9 months, 12 months, 15 months, 18 months, 2 years or more than 2 years.

In some embodiments of the methods of this disclosure, the bDMARD is administered in combination with an additional agent, such as an anti-rheumatic drug. In some embodiments, the additional agent coadministered is an anti-rheumatic drug that is a non-biological DMARD, such as e.g. methotrexate or leflunomide, or a NSAID.

In some embodiments of the methods of this disclosure, the patient is refractory to one or more conventional therapies. In some embodiments of the methods of this disclosure, the patient has failed to respond to treatment with a bDMARD having an Fc fragment, e.g a TNFα inhibitor, such as adalimumab, infliximab, etanercept, or golimumab; a T cell costimulatory inhibitor, such as abatacept; an IL-6 or IL-6 receptor inhibitor, such as tocilizumab, sarilumab or olokizumab, or an anti-CD20 antibody, such as rituximab; or anakinra; or belimumab. In some embodiments of the methods of this disclosure, the patient has failed to respond to treatment with a non-biological DMARD. In some embodiments, the DMARD is methotrexate, leflunomide, sulfasalazine, chloroquine, hydroxychloroquine, gold, azathioprine, ciclosporin, mycofenolate mofetil or cyclophosphamide.

### Definitions

The term “at baseline” as used herein refers to the moment of therapeutic decision, e.g. when a patient has been assessed by a physician and a treatment decision is made.

The term “CDAI” as used herein refers to the clinical disease activity index (CDAI), a composite measure of disease activity in RA that integrates swollen joint count, tender joint count, patient global assessment of disease activity and physician global assessment of disease activity (Takanashi et al., CDAI and DAS28 in the management of rheumatoid arthritis in clinical practice, Ann Rheum Dis. 2020).

The term “DAS28-CRP” as used herein refers to a disease activity score (DAS) in RA that integrates tender and swollen joint counts (28 joints are systematically evaluated), patient global assessment of disease activity and serum CRP concentrations (Takanashi et al., CDAI and DAS28 in the management of rheumatoid arthritis in clinical practice, Ann Rheum Dis. 2020 and Greenmyer et al., DAS28-CRP Cutoffs for High Disease Activity and Remission Are Lower Than DAS28-ESR in Rheumatoid Arthritis, ACR Open Rheumatol. 2020).

The term “disease modifying anti-rheumatic drug(s) [DMARD(s)]” as used herein refers to are a group of drugs commonly used in patients with rheumatic diseases, particularly rheumatoid arthritis but also ankylosing spondylitis, juvenile idiopathic arthritis, psoriatic arthritis, and systemic lupus erythematosusas, as known in the art. DMARDs include non-bioligical DMARDs, such as methotrexate, sulfasalazine, leflunomide, antimalarial drugs (e.g. chloroquine or hydroxychloroquine), azathioprine and cyclosporine as well as biological DMARDs (bDMARDs), such as etanercept, infliximab, adalimumab, certolizumab pegol, golimumab, abatacept, rituximab, tocilizumab, sarilumab, olokizumab, anakinra and belimumab.

The term “failed to respond to treatment” as used herein refers to failure to reach the treatment target, i.e. low disease activity (LDA).

The term “having an Fc fragment” as used herein refers to a molecule which comprises the polypeptide chain of an immunoglobulin Fc fragment, e.g. a full length antibody or an antibody derivate such as etanercept.

The term “lacking an Fc fragment” as used herein refers to a molecule which does not comprise the polypeptide chain of an immunoglobulin Fc fragment. Such molecule can be a peptide or polypeptide, e.g. an antibody derivative, such as Fab, modified Fab, Fab′, modified Fab′, F(ab′) 2, Fv, Fab-Fv, Fab-dsFv, Fab-Fv-Fv, scFv and Bis-scFv fragment. Said fragment can also be a diabody, tribody, TrYbe™, triabody, tetrabody, minibody, single domain antibody (dAb) such as sdAb, VL, VH, VHH or camelid antibody (e.g. from camels or llamas such as a Nanobody™) and VNAR fragment or other fragment of an antibody lacking the Fc fragment. The molecule can be modified, e.g. by attachment of polyethylene glycol (PEG). A molecule which does not comprise the polypeptide chain of an immunoglobulin Fc fragment can be a DMARD, e.g. a biological DMARD or a non-biological DMARD. Examples of a molecule which does not comprise the polypeptide chain of an immunoglobulin Fc fragment are certolizumab pegol and dapirolizumab pegol.

The term “loading dose” as used herein refers to drug doses used at initiation of therapy to ensure that plasma drug concentrations quickly reach the therapeutic range.

The term “low disease activity or LDA” as used herein refers to treatment targets in RA (CDAI≤10, DAS28-ESR≤3.2 or DAS28-CRP≤3.2), JIA (juvenile idiopathic arthritis DAS27≤3.8), SLE (1. SLE Disease Activity Index (SLEDAI)-2K≤4, with no activity in major organ systems (renal, central nervous system (CNS), cardiopulmonary, vasculitis, fever) and no hemolytic anemia or gastrointestinal activity; 2. no new lupus disease activity compared with the previous assessment; 3. a Safety of Estrogens in Lupus Erythematosus National Assessment (SELENA)-SLEDAI physician global assessment (scale 0-3)≤1; 4. a current prednisolone (or equivalent) dose≤7.5 mg daily; and 5. well tolerated standard maintenance doses of immunosuppressive drugs and approved biological agents).

The term “maintenance dose” as used herein refers to drug doses used chronically, e.g. by long term, repeated administration, to ensure that plasma drug concentrations are maintained in the therapeutic range.

The term “no efficacy response” as used herein refers to the inability to reach the treatment target (e.g. LDA).

The term “primary failure” as used herein refers to the inability to reach the treatment target (e.g. LDA) within 12 to 18 months of therapy, pointing at the inability of the drug to suppress disease activity.

The term “remission” as used herein refers to the resolution of disease symptoms, e.g. in RA inflammatory symptoms (DAS28-CRP≤2.6 in RA).

The term “SDAI” as used herein refers to the simplified disease activity index (SDAI), a composite measure of disease activity in RA that integrates swollen joint count, tender joint count, serum CRP, patient global assessment of disease activity and physician global assessment of disease activity (Smolen et al., A simplified disease activity index for rheumatoid arthritis for use in clinical practice, Rheumatology (Oxford), 2003).

The term “secondary failure” as used herein refers to the loss of the ability of the drug to reach the treatment target (e.g. LDA) after a initially ability to reach the treatment target (e.g. LDA) within 12 to 18 months of therapy. Often the secondary failure of a bDMARD is due to the development of anti-drug antibodies.

The term “seropositive for rheumatoid factor” as used herein refers to the presence of rheumatoid factors above the upper value of normal (the definition of which differs for each measurement technique or test) in at least one serum sample of a patient (Ingegnoli et al., Rheumatoid Factors: Clinical Applications, Dis Markers. 2013 and Trier et al, Determination of Rheumatoid Factors by ELISA, Methods Mol Biol, 2019).

The term “substantial clinical benefit” as used herein refers to an improvement in clinical symptoms that exceeds the minimal clinically significant difference (e.g. delta CDAI>1, in RA when the baseline CDAI≤is 10).

### Additional Embodiments

This disclosure is further described by the following non-limiting clauses:
Clause 1. A method of treating a rheumatic disease in a human patient having rheumatoid factor comprising:
administering a bDMARD lacking an Fc fragment to a human patient identified as having rheumatoid factor above 100 IU/ml, above 121.4 IU/ml, above 125 IU/ml, above 145.9 IU/ml, above 150 IU/ml, above 175 IU/ml, above 200 IU/ml or above 204 IU/ml in serum;
wherein (i) the therapeutic effect of the bDMARD lacking an Fc fragment is not reduced by RF in said patient and/or (i) the treatment induces low disease activity or achieves substantial clinical benefit in the patient.
Clause 2. A method of treating a rheumatic disease in a human patient having rheumatoid factor comprising:
determining the titer of rheumatoid factor in a sample, preferably a serum sample, from said patient;
administering a bDMARD lacking an Fc fragment to said patient if the titer of rheumatoid factor is above 100 IU/ml, above 121.4 IU/ml, above 125 IU/ml, above 145.9 IU/ml, above 150 IU/ml, above 175 IU/ml, above 200 IU/ml, above 204 IU/ml, above 225 IU/ml, above 250 IU/ml, or above 275 IU/ml in serum;
wherein (i) the therapeutic effect of the bDMARD lacking an Fc fragment is not reduced by RF in said patient and/or (i) the treatment induces low disease activity or achieves substantial clinical benefit in the patient.
Clause 3. A method of treating rheumatoid arthritis in a human patient having rheumatoid factor comprising:
administering certolizumab pegol to a human patient identified as having rheumatoid factor above 100 IU/ml, above 121.4 IU/ml, above 125 IU/ml, above 145.9 IU/ml, above 150 IU/ml, above 175 IU/ml, above 200 IU/ml or above 204 IU/ml in serum;
wherein (i) the therapeutic effect of certolizumab pegol is not reduced by RF in said patient and/or (i) the treatment induces low disease activity or achieves substantial clinical benefit in the patient.
Clause 4. A method of treating rheumatoid arthritis in a human patient having rheumatoid factor comprising:
determining the titer of rheumatoid factor in a sample, preferably a serum sample, from said patient;
administering certolizumab pegol to said patient if the titer of rheumatoid factor is above 100 IU/ml, above 121.4 IU/ml, above 125 IU/ml, above 145.9 IU/ml, above 150 IU/ml, above 175 IU/ml, above 200 IU/ml, above 204 IU/ml, above 225 IU/ml, above 250 IU/ml, or above 275 IU/ml in serum;
wherein (i) the therapeutic effect of certolizumab pegol is not reduced by RF in said patient and/or (i) the treatment induces low disease activity or achieves substantial clinical benefit in the patient.
Clause 5. The method according to any one of clauses 1-4, wherein the low disease activity is achieved 90 days, 100 days, 104 days, 120 days or 180 days after the first administration of certolizumab pegol.
Clause 6. The method according to any one of clauses 1-5, wherein the low disease activity in the patient is achieved with a likelihood of 40%, 45%, 50% or 55%.
Clause 7. The method according to any one of clauses 3-6 wherein certolizumab pegol is administered to the patient in step (b) at loading dose of 400 mg initially and 400 mg two weeks later followed by a maintenance dose of (i) 400 mg of every 4 weeks or (ii) 200 mg every 2 weeks.
Clause 8. The method according to any one of clauses 1-7, wherein no loading dose is administered to the patient at the initiation of treatment.
Clause 9. The method according to any one of clauses 1-8, wherein the rheumatoid factor is of the IgM, IgG, IgD, IgE or IgA isotype.
Clause 10. The method according to any one of clauses 3-9, wherein certolizumab pegol is administered in combination with another anti-rheumatic drug.
Clause 11. The method according to clause 10, wherein the anti-rheumatic drug is methotrexate or leflunomide.
Clause 12. The method according to any one of clauses 1-11, wherein the patient has previously failed to respond to treatment with a DMARD.
Clause 13. The method according to clause 12, wherein the patient has previously failed to respond to treatment with a biological DMARD having an Fc fragment.
Clause 14. The method according to clause 12, wherein the patient has previously failed to respond to treatment with a non-biological DMARD.
Clause 15. The method according to clauses 13 or 14, wherein treatment failure is a primary failure.
Clause 16. The method according to clauses 13 or 14, wherein treatment failure is a secondary failure.
Clause 17. The method according to any one of clauses 1-16, wherein bDMARD lacking an Fc fragment is administered at regular intervals for at least 3 months.
Clause 18. The method according to any one of clauses 1-17, wherein the plasma concentration of the bDMARD lacking an Fc fragment is not decreased in the patient following its administration.:
Clause 19. A method of maintaining low disease activity or remission of a rheumatic disease in a human subject in need thereof, the method comprising:
administering to the subject a dose of a bDMARD lacking an Fc fragment that is effective to maintain low disease activity or remission in the subject,
wherein the subject is identified as having an elevated baseline rheumatoid factor (RF) serum level of more than 100 IU/ml.
Clause 20. The method of clause 19, wherein the subject has rheumatoid arthritis (RA).
Clause 21. The method of clause 20, wherein the subject has early RA.
Clause 22. The method of clause 20, wherein the subject had active RA for at least 6 months prior to treatment.
Clause 23. The method of any one of clauses 19-22, wherein the bDMARD is certolizumab pegol.
Clause 24. The method of clause 23, wherein the certolizumab pegol is administered to the subject at a loading dose of 400 mg initially and 400 mg two weeks later followed by a maintenance dose.
Clause 25. The method of clause 23, wherein no loading dose is administered to the patient at the initiation of treatment with a maintenance dose.
Clause 26. The method of clause 25, wherein the certolizumab pegol is administered to the subject at a maintenance dose of 400 mg of every 4 weeks.
Clause 27. The method of clause 25, wherein the certolizumab pegol is administered to the subject at a maintenance dose of 200 mg every 2 weeks.
Clause 28. The method of any one of clauses 19-27, wherein the bDMARD is administered to the subject for at least 12 weeks.
Clause 29. The method of any one of clauses 19-28, wherein the bDMARD is administered to the subject for at least 24 weeks.
Clause 30. The method of any one of clauses 19-29, wherein the bDMARD is administered to the subject for at least 1 year.
Clause 31. The method of any one of clauses 19-30, wherein the bDMARD is administered to the subject for at least 2 years.
Clause 32. The method of any one of clauses 19-31, wherein low disease activity or remission is achieved by 12 weeks after the first administration of the bDMARD, and maintained during the period of administration.
Clause 33. The method of any one of clauses 19-32, wherein the subject is identified as having a baseline RF serum level of more than 121.4 IU/ml.
Clause 34. The method of any one of clauses 19-32, wherein the subject is identified as having a baseline RF serum level of more than 125 IU/ml.
Clause 35. The method of any one of clauses 19-32, wherein the subject is identified as having a baseline RF serum level of more than 145.9 IU/ml.
Clause 36. The method of any one of clauses 19-32, wherein the subject is identified as having a baseline RF serum level of more than 150 IU/ml.
Clause 37. The method of any one of clauses 19-32, wherein the subject is identified as having a baseline RF serum level of more than 175 IU/ml.
Clause 38. The method of any one of clauses 19-32, wherein the subject is identified as having a baseline RF serum level of more than 200 IU/ml.
Clause 39. The method of any one of clauses 19-32, wherein the subject is identified as having a baseline RF serum level of more than 204 IU/ml.
Clause 40. The method of any one of clauses 19-32, wherein the subject is identified as having a baseline RF serum level of more than 225 IU/ml.
Clause 41. The method of any one of clauses 19-32, wherein the subject is identified as having a baseline RF serum level of more than 250 IU/ml.
Clause 42. The method of any one of clauses 19-32, wherein the subject is identified as having a baseline RF serum level of at least 275 IU/ml.
Clause 43. The method of any one of clauses 19-43, further comprising the earlier step of determining the serum level of RF of a subject having a rheumatic disease.
Clause 44. The method of any one of clauses 19-44, wherein the RF is of the IgM, IgG, IgD, IgE or IgA isotype.
Clause 45. The method of any one of clauses 19-44, wherein the bDMARD is administered in combination with another anti-rheumatic drug.
Clause 46. The method according to clause 45, wherein the anti-rheumatic drug is methotrexate or leflunomide.

### EXAMPLES

### Example 1

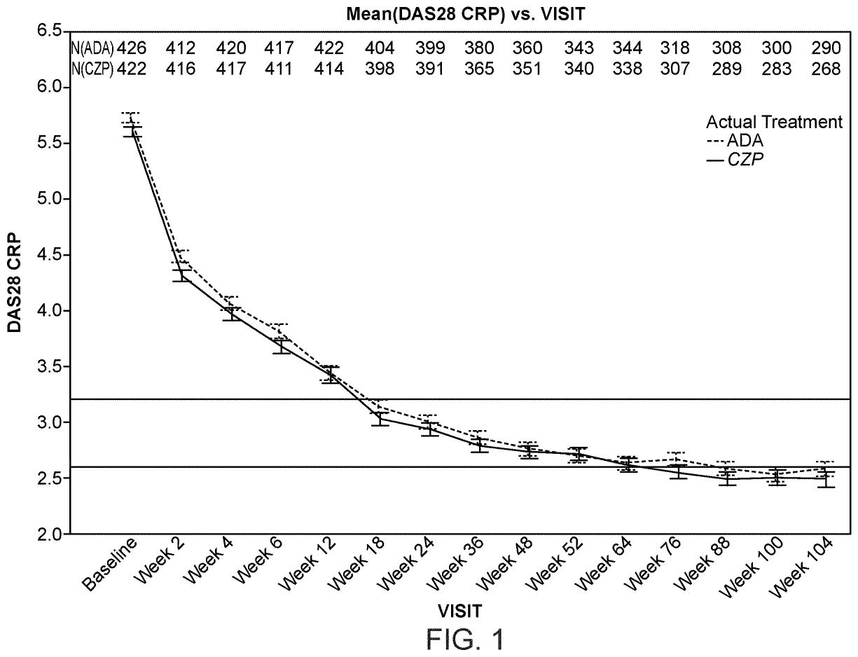

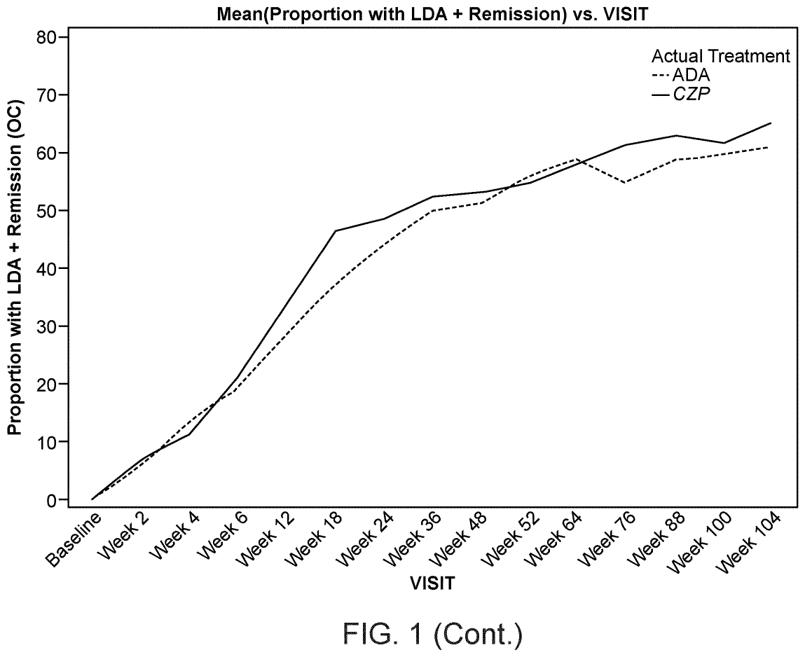

We performed post-hoc analyses of EXXELERATE, a phase 4 trial comparing two TNF inhibitors, adalimumab and certolizumab pegol in combination with methotrexate in patients with rheumatoid arthritis and moderate-to-severe disease activity. By opposition to adalimumab that is a full monoclonal antibody, certolizumab pegol is a PEGylated Fab fragment of an antibody Thus, certolizumab pegol does not have an Fc fragment. 453 patients were randomized to certolizumab pegol and 454 were randomized to adalimumab. Both treatments resulted in similar effects in terms of efficacy (FIG. 1) and tolerance.

In our post-hoc analyses, we studied response to therapy (i.e. mean DAS28-CRP, mean DAS28-ESR, mean CDAI, mean SDAI, proportions of patients achieving DAS28-CRP low disease activity (LDA) or disease remission) and drug plasma concentrations until week 104 in patients stratified by RF titer quartile (Q1: 0-32 IU/mL, Q2: 32-75 IU/mL, Q3: 75-204 IU/mL, Q4: >204 IU/mL). Baseline characteristics showed that disease activity, HAQ, ACPA titers were higher in patients in RF Q4, in line with the well-known association between high titers RF and disease severity. In each quartile, there were no differences in baseline characteristics of certolizumab pegol-versus adalimumab-treated patients.

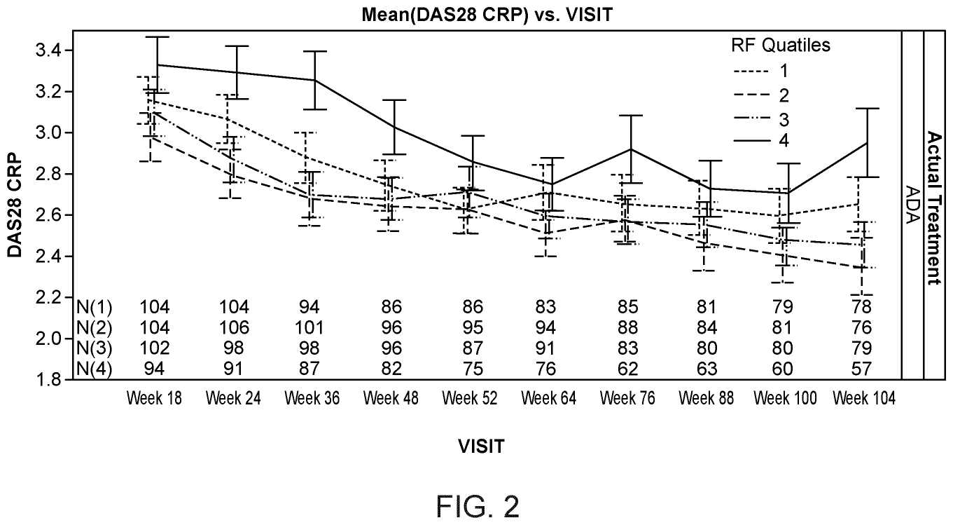

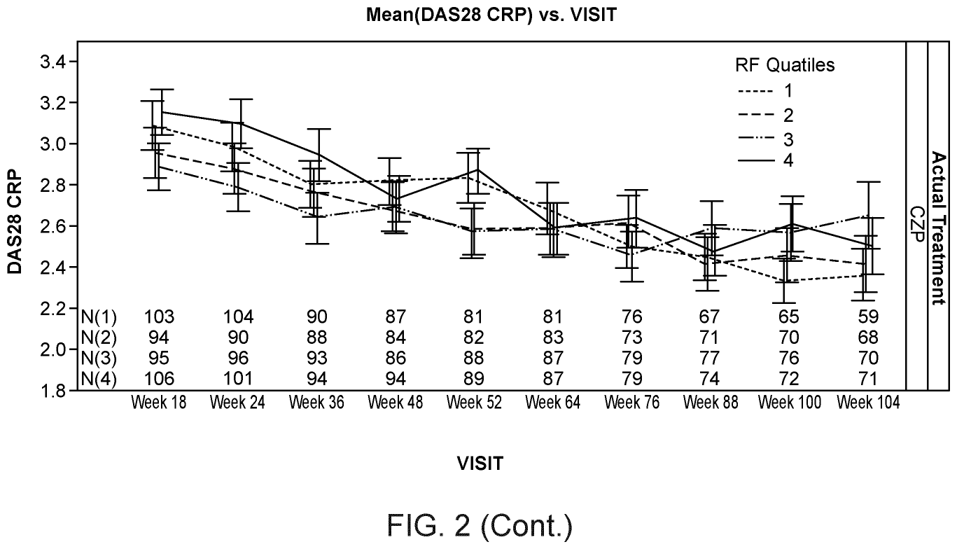

In adalimumab-treated patients, we found as expected that measures of disease activity progressed less favorably in patients with high compared to low RF titers. Thus, mean DAS28-CRP values over time were higher in response to adalimumab in RF Q4 compared to RF Q1-3 patients [FIG. 2]. By contrast, such difference was not observed in certolizumab pegol-treated patients: mean DAS28-CRP progression over time was similar in patients with high or low RF titers, indicating that efficacy of certolizumab pegol was not influenced by RF titers in these patients [FIG. 2 (Cont.)].

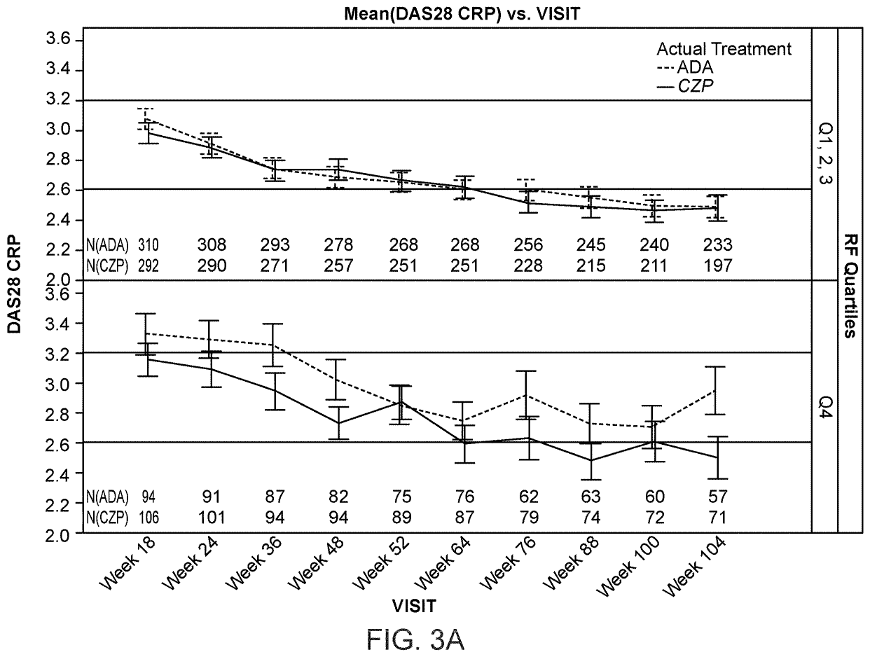

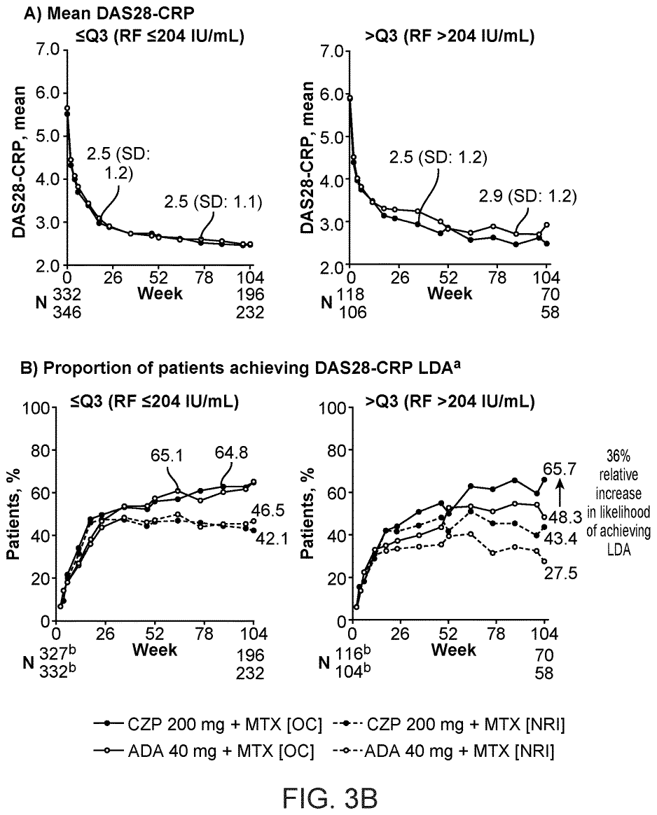

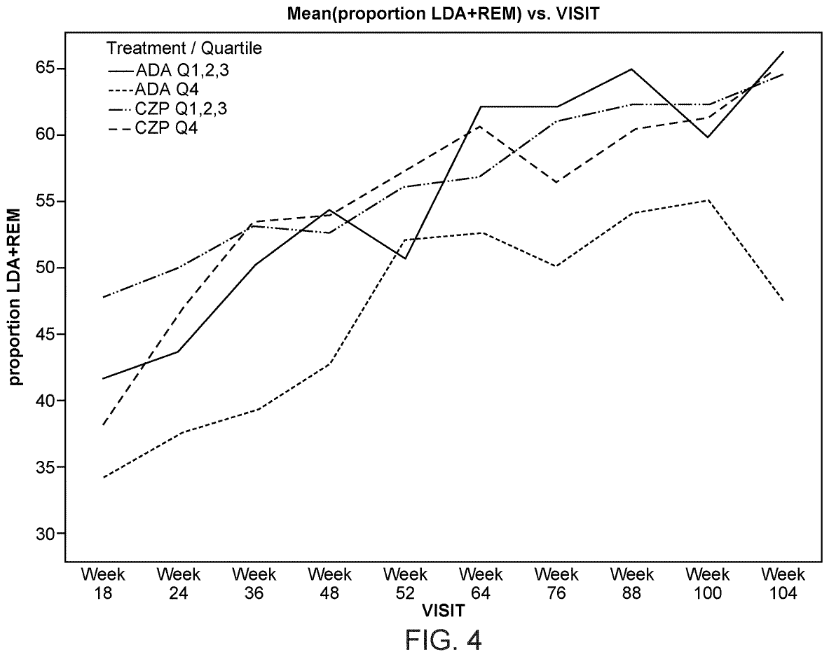

Hence, DAS28-CRP scores were superposed for certolizumab pegol and adalimumab in patients with low RF titers. But in patients with high titers RF, DAS28-CRP scores were generally lower in certolizumab pegol-versus adalimumab-treated patients, which is indicative of stronger clinical improvement in response to certolizumab pegol in this category of patients with severe disease [FIGS. 3A-3B]. Similar patterns were observed for all disease activity outcome measures (DAS28-ESR, CDAI, SDAI). Looking at proportions of patients reaching low disease activity or remission, we observed that these rates were similar for certolizumab pegol and adalimumab in patients with low RF titers. Proportions of patients in low disease activity or remission were lower for adalimumab in patients with high RF titers. However, this was surprisingly not the case for certolizumab pegol, in which proportions of patients in low disease activity or remission were similar in patients with high versus low RF titers. These differences between certolizumab pegol and adalimumab were observed in both the ‘per protocol’ and ‘intention to treat’ analyses of the data (FIG. 4).

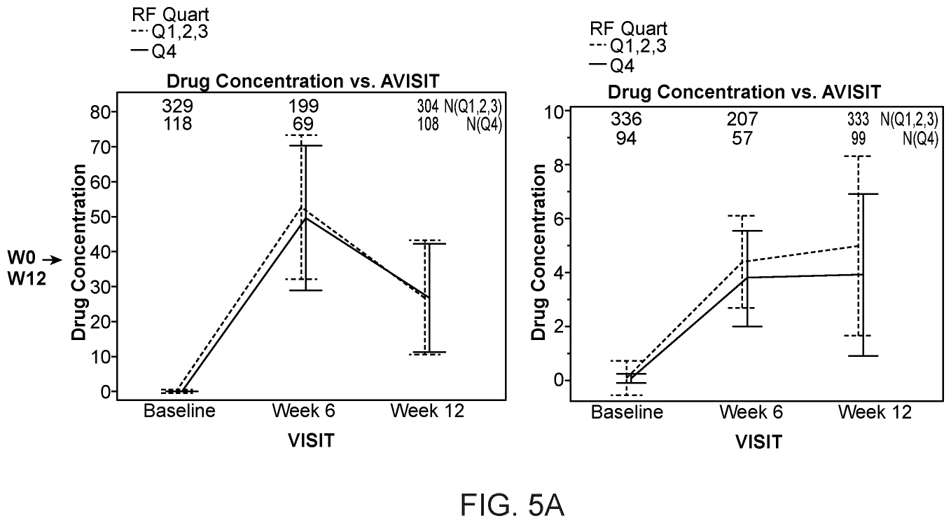

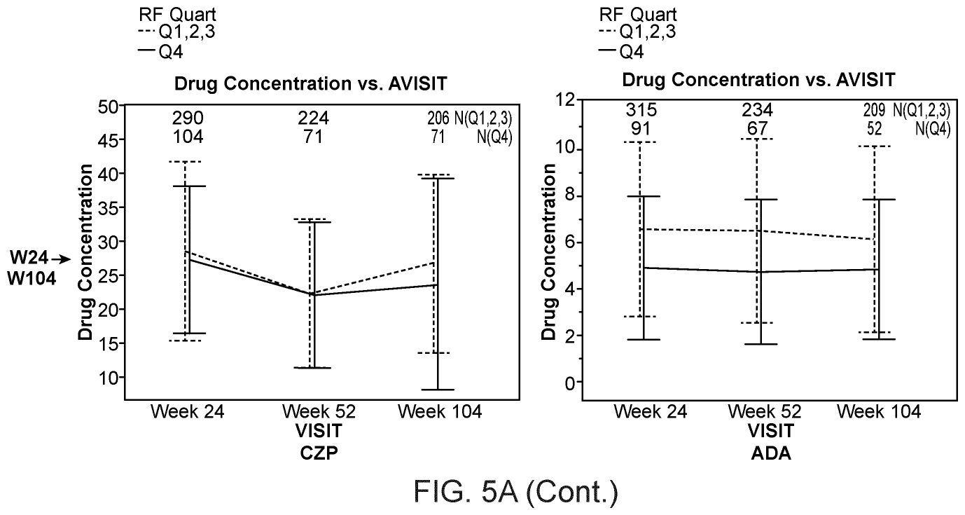

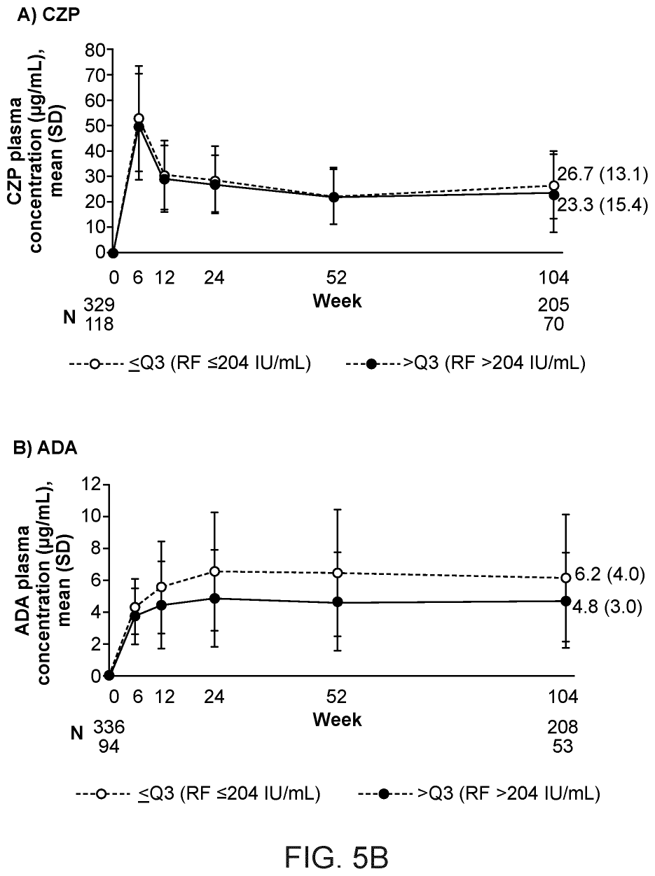

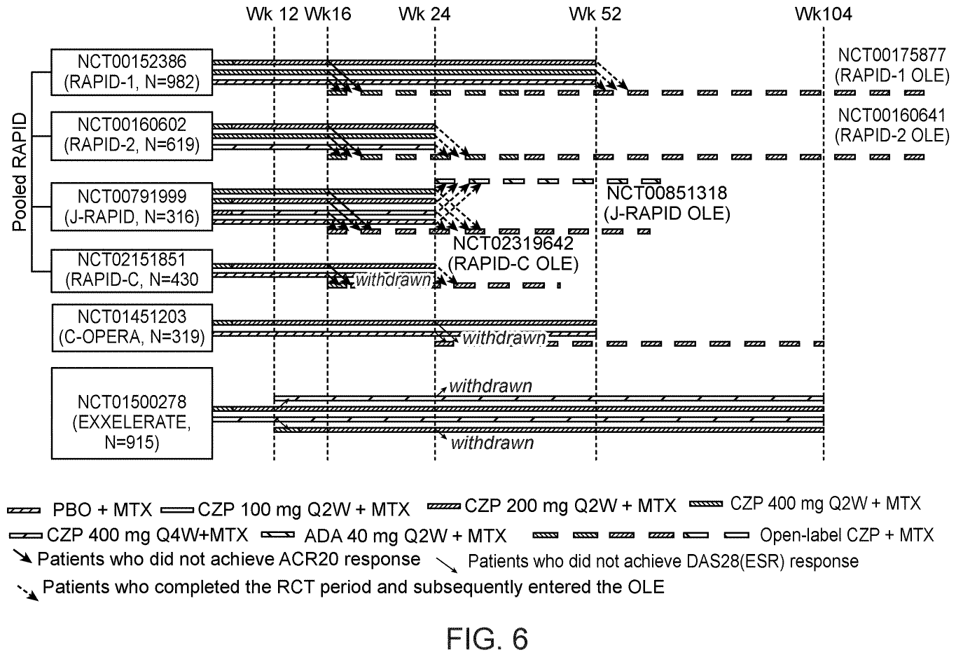

In parallel with these different patterns of response to therapy between certolizumab pegol and adalimumab in patients with high versus low titers RF, we observed differences in patterns of plasma drug concentrations between both biologics. Thus, mean adalimumab drug concentrations were lower in patients with high compared to low titers RF. By contrast, this difference was not observed with certolizumab pegol that maintained similar drug concentrations in patients with high compared to low RF titers [FIG. 5A].

Taken together, our observations indicate that certolizumab pegol does not behave as expected in patients with high titers RF. By opposition to adalimumab, certolizumab pegol maintains therapeutic responses and plasma drug concentrations in patients with high titers RF, similar to these observed in patients with low titers RF.

### Example 2

### Study Design

This post-hoc analysis included data from six clinical trials of certolizumab pegol in patients with RA: C-OPERA (NCT01451203), RAPID-1 (NCT00152386), RAPID-2 (NCT00160602), J RAPID (NCT00791999), RAPID-C(NCT02151851) and EXXELERATE (NCT01500278). The study designs of the trials are presented in FIG. 1. The full study designs, patient populations, and results of all six clinical studies have been published previously. Data from RAPID-1, RAPID-2, J RAPID, and RAPID-C were pooled for this study.

### Study Participants

C-OPERA included Japanese patients with early RA. Briefly, methotrexate (MTX)-naïve patients aged 20-64 years with active RA≤12 months (defined by 2010 ACR/EULAR classification criteria) were randomized to receive placebo (PBO) Q2W plus methotrexate (PBO+MTX) or certolizumab pegol 200 mg Q2W (loading dose of certolizumab pegol 400 mg at Weeks 0/2/4) plus MTX (certolizumab pegol+MTX) for 52 weeks.

Pooled RAPID included patients from two global trials (RAPID-110 and RAPID 211), a Japanese trial (J-RAPID), and a Chinese trial (RAPID-C). Briefly, patients≥18 years (>20 years for J-RAPID) with active RA for ≥6 months (defined by ACR 1987 criteria), who received MTX for ≥6 months (≥3 months for RAPID C) prior to baseline, were randomized to receive PBO+MTX or certolizumab pegol 400 mg Q2W plus MTX or certolizumab pegol 200 mg Q2W (loading dose of certolizumab pegol 400 mg at Weeks 0/2/4) plus MTX (certolizumab pegol+MTX) for at least 24 weeks (52 weeks for RAPID-1, 24 weeks for RAPID-2, J RAPID, and RAPID-C).

EXXELERATE was a head-to-head superiority study comparing certolizumab pegol with ADA in patients with active RA with prognostic factors for severe disease progression. Briefly, patients≥18 years with active RA (defined by 2010 ACR/EULAR classification criteria) were randomized to receive certolizumab pegol 200 mg Q2W (loading dose of certolizumab pegol 400 mg at Weeks 0/2/4) plus MTX (certolizumab pegol+MTX), or ADA 40 mg Q2W plus MTX (ADA+MTX) for 104 weeks.

### Assessment of Rheumatoid Factor Level

RF levels (IgM RF) were measured using validated immunoassays in local hospitals, by nephelometry or enzyme-linked immunosorbent assay (ELISA). There is a good correlation between the two methods of RF quantification despite potential variations in cut-off levels; both methods are adequately sensitive for routine laboratory purposes.26 Patients were classified into quartiles (Q) based on overall baseline RF levels of each study. For C-OPERA, the RF quartiles were defined as <59.0 IU/mL, ≥59.0-<93.0 IU/mL, ≥93.0-<275.0 IU/mL, and ≥275.0 IU/mL for Q1-4, respectively. For Pooled RAPID, the RF quartiles were defined as <25.0 IU/mL, ≥25.0-<78.5 IU/mL, ≥78.5-<207.0 IU/mL, and ≥207.0 IU/mL for Q1-4, respectively. For EXXELERATE, the RF quartiles were defined as ≤32.0 IU/mL, >32.0-≤75.0 IU/mL, >75.0-≤204.0 IU/mL, and >204 IU/mL for Q1-4, respectively.

### Outcomes

The efficacy of certolizumab pegol was assessed by DAS28-erythrocyte sedimentation rate (DAS28 [ESR]); efficacy assessments (mean DAS28 [ESR]) were performed through Weeks 0-24. In this analysis, DAS28 (ESR) 2.6-≤3.2 was classified as low disease activity (LDA) and DAS28 (ESR)<2.6 was classified as remission (REM). The proportion of patients who achieved DAS28 (ESR) LDA and REM were evaluated at Week 12 and Week 24.

### Statistical Analysis

All analyses were performed using the full analysis set, unless otherwise stated. The full analysis set included all patients who received≥1 dose of study drug and provided any efficacy data thereafter. Observed data for DAS28 (ESR) response are reported. As this was a post-hoc subgroup analysis, all data reported are descriptive only.

### Results

### Patient Demographics and Baseline Characteristics

In C-OPERA, 316 patients (certolizumab pegol+MTX n=159; PBO+MTX n=157) were included in this post-hoc analysis. In Pooled RAPID, 1,537 patients (certolizumab pegol+MTX n=1,025; PBO+MTX n=512) were included in this post-hoc analysis. In EXXELERATE, 908 patients (certolizumab pegol+MTX n=454; ADA+MTX n=454) were included in the full analysis set which was used for the analysis of baseline demographics and Weeks 0-12 efficacy outcomes in this post hoc analysis. For Weeks 12-24, the Week 12 full analysis set (certolizumab pegol+MTX n=352; ADA+MTX n=361), consisting of certolizumab pegol+MTX patients randomized to certolizumab pegol who responded at Week 12 and continued on certolizumab pegol and ADA+MTX patients randomized to ADA who responded at Week 12 and continued on ADA, was analyzed.

At baseline, patient demographics and disease characteristics were similar between treatment groups for C-OPERA, Pooled RAPID, and EXXELERATE. Within each treatment group (PBO+MTX or certolizumab pegol+MTX or ADA+MTX), mean DAS28 (ESR) scores at baseline were similar across RF quartiles for C-OPERA, Pooled RAPID, and EXXELERATE.

### DAS28 (ESR) LDA and REM Rates

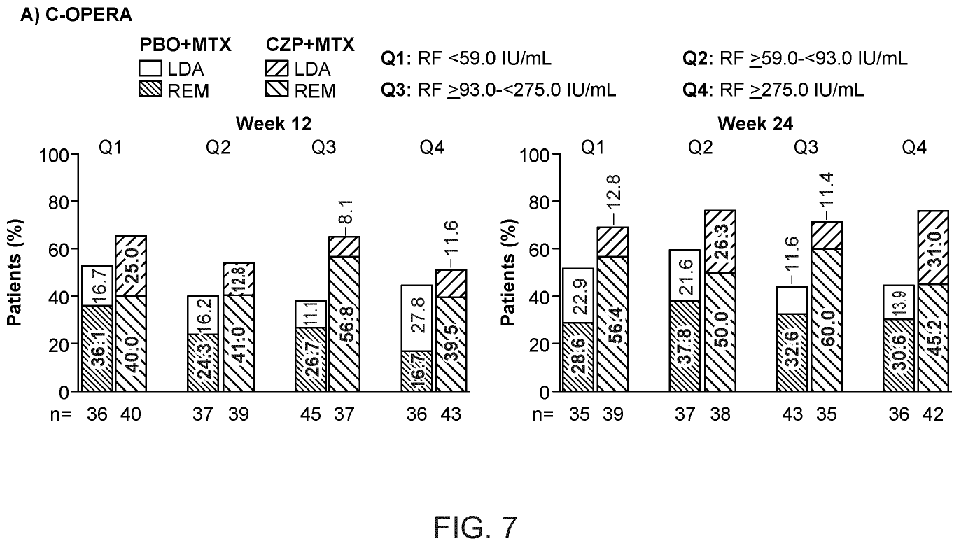

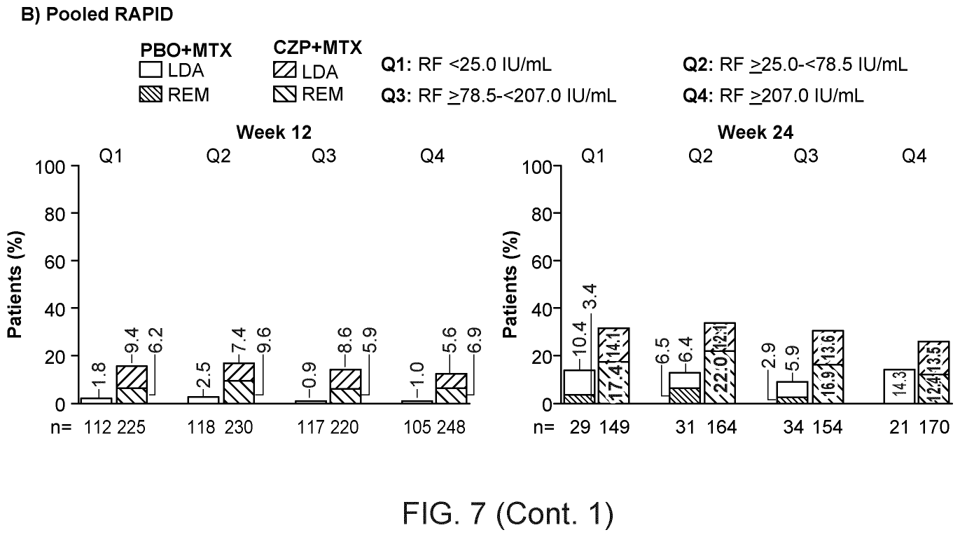

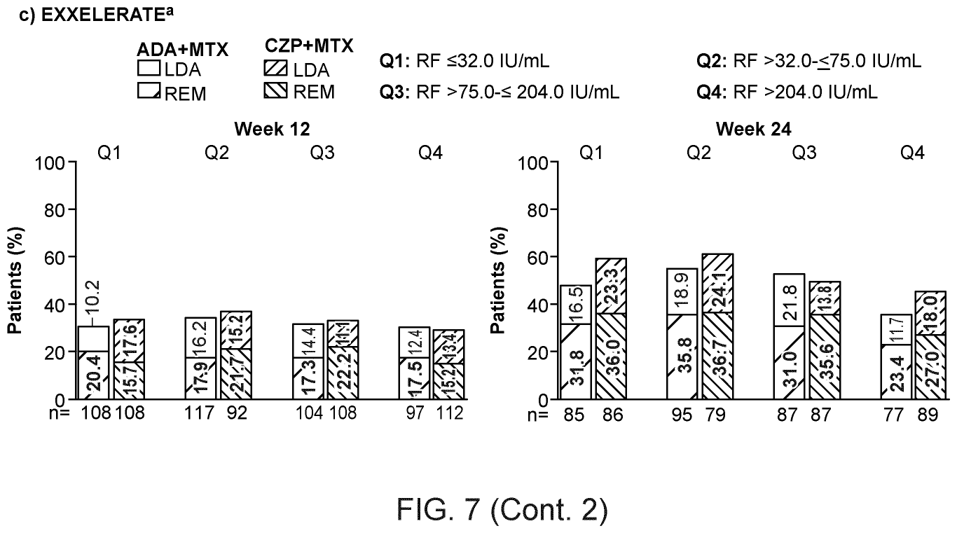

For C-OPERA, DAS28 (ESR) LDA and REM rates were numerically higher in the certolizumab pegol+MTX group compared with the PBO+MTX group at Weeks 12 and 24, across RF quartiles. In the certolizumab pegol+MTX group, DAS28 (ESR) LDA and REM rates improved from Week 12 to Week 24, across RF quartiles. At Weeks 12 and 24, DAS28 (ESR) LDA and REM rates were comparable across RF quartiles in the certolizumab pegol+MTX group [FIG. 7, panel (a)].

For Pooled RAPID, DAS28 (ESR) LDA and REM rates were numerically higher in the certolizumab pegol+MTX group compared with the PBO+MTX group at Weeks 12 and 24, across RF quartiles. In the certolizumab pegol+MTX group, DAS28 (ESR) LDA and REM rates improved from Week 12 to Week 24, across RF quartiles. At Weeks 12 and 24, DAS28 (ESR) LDA and REM rates were comparable across RF quartiles in the certolizumab pegol+MTX group [FIG. 7, panel (b)].

For EXXELERATE, DAS28 (ESR) LDA and REM rates were similar in certolizumab pegol+MTX compared to ADA+MTX at Weeks 12 and 24, across all RF quartiles. In both certolizumab pegol+MTX and ADA+MTX groups, DAS28 (ESR) LDA and REM rates improved from Week 12 to Week 24, across RF quartiles [FIG. 7, panel (c)]. In the certolizumab pegol+MTX group, this improvement was consistent across RF quartiles. In the ADA+MTX group, the improvement was numerically lower in the subgroup with the highest RF levels (Q4) compared with other RF quartiles.

### Mean DAS28 (ESR) Over 24 Weeks

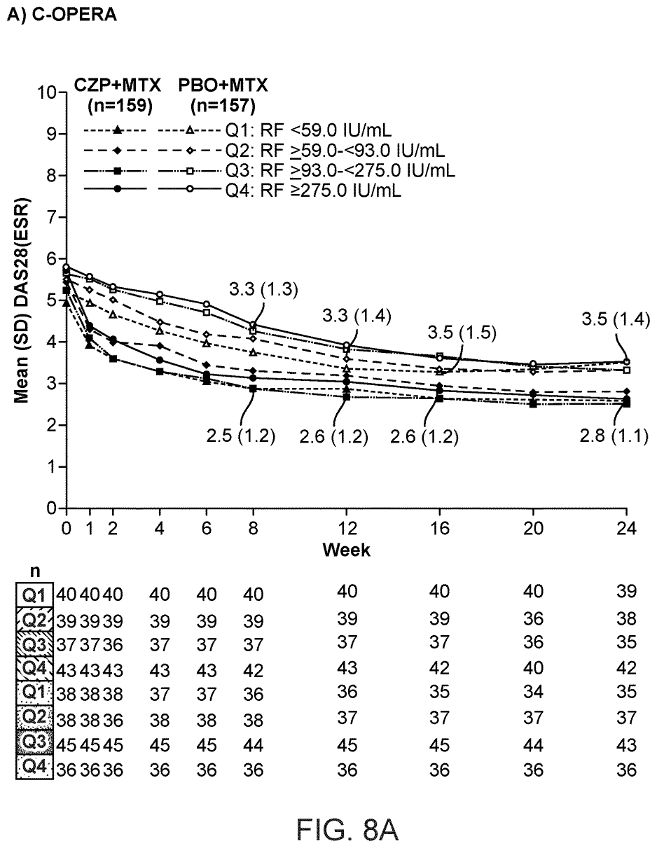

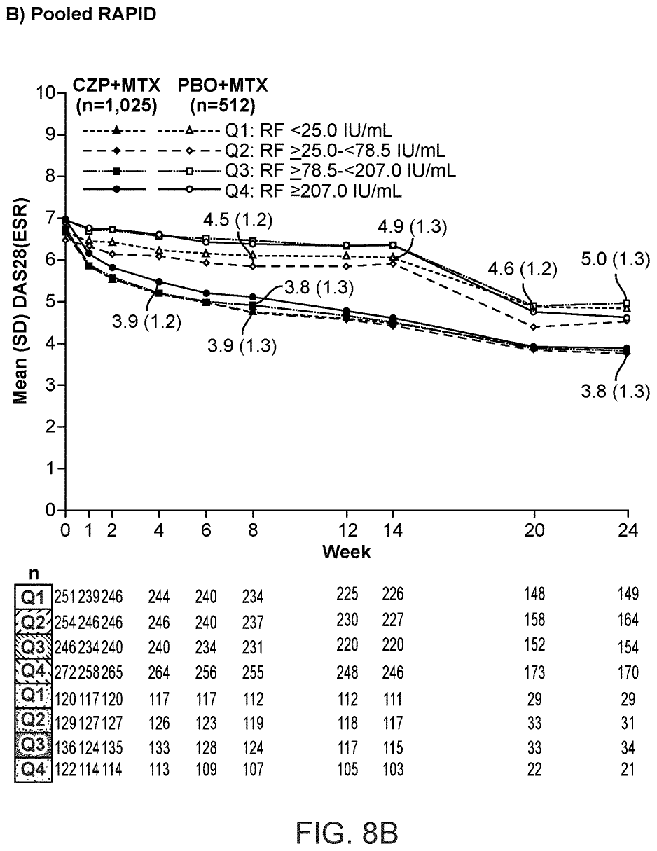

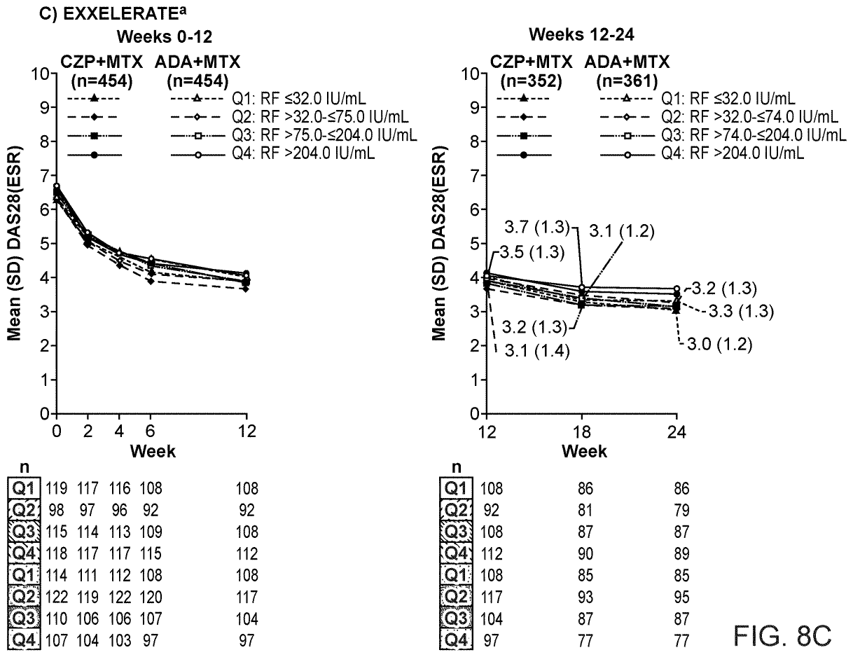

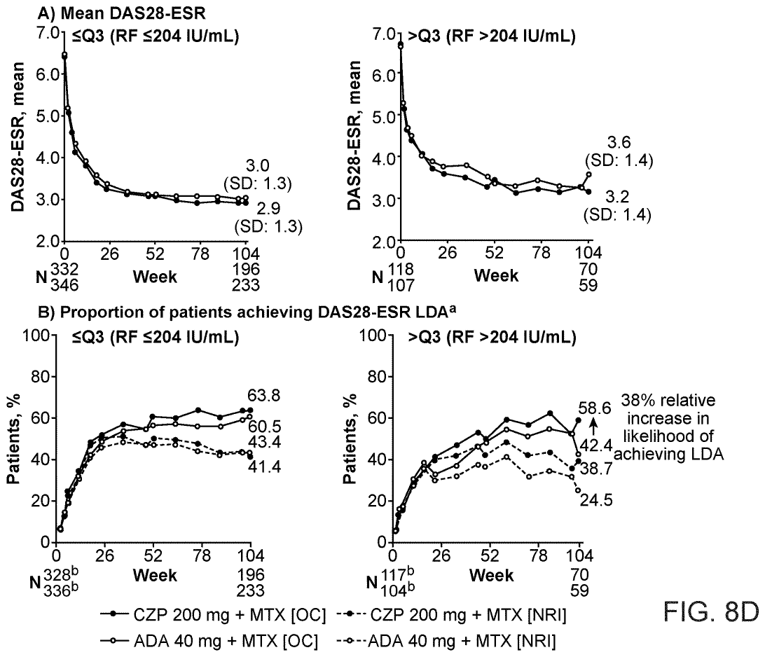

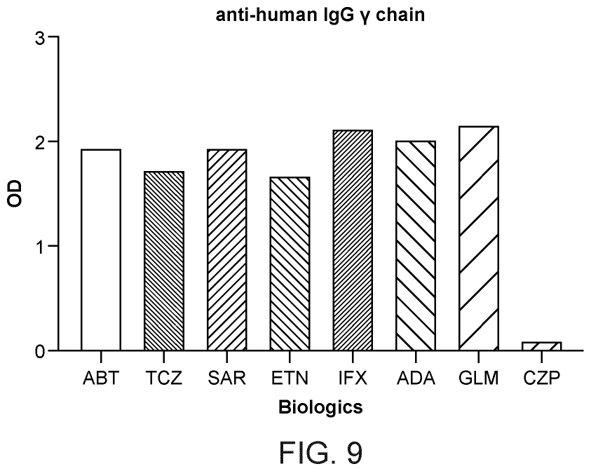

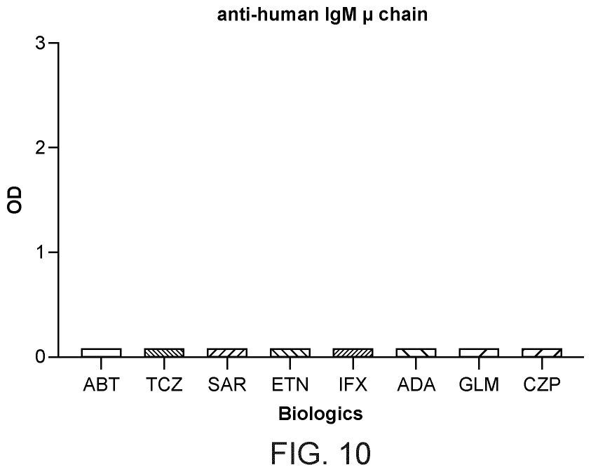

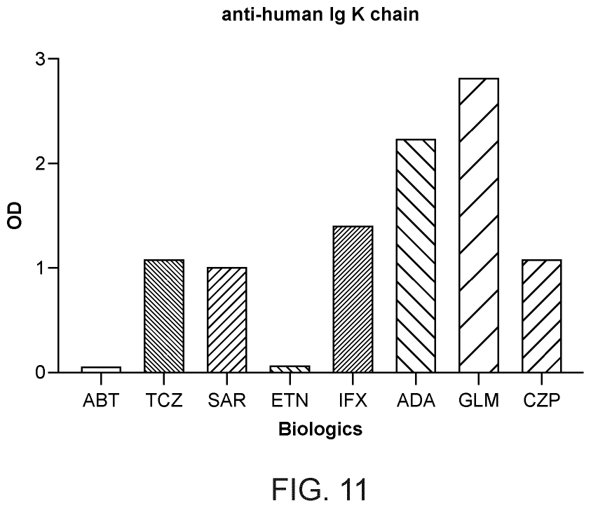

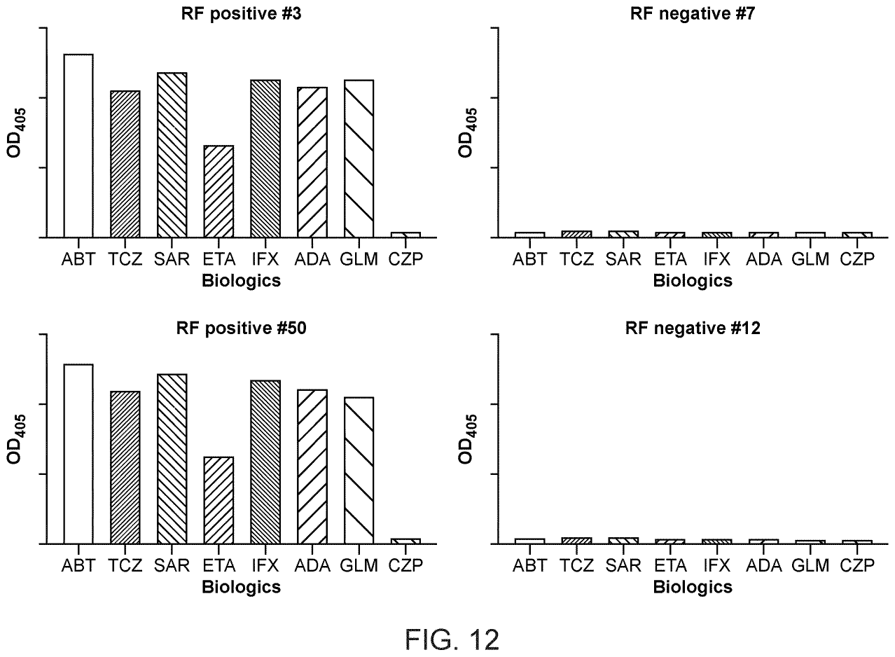

For C-OPERA and Pooled RAPID, in both certolizumab pegol+MTX and PBO+MTX groups, mean DAS28 (ESR) decreased from Week 0 to Week 24, across RF quartiles [FIG. 8A and (b), respectively]. Mean DAS28 (ESR) was numerically lower in certolizumab pegol+MTX compared with PBO+MTX over 24 weeks, across RF quartiles.

For EXXELERATE, in both certolizumab pegol+MTX and ADA+MTX groups, mean DAS28 (ESR) decreased from Weeks 0-12 and Weeks 12-24 across RF quartiles [FIG. 8C]. Mean DAS28 (ESR) for certolizumab pegol+MTX and ADA+MTX groups were similar over 24 weeks, across RF quartiles.

### Discussion

In this post-hoc analysis, efficacy of certolizumab pegol was surprisingly comparable across baseline RF subgroups over 24 weeks, in patients with early RA (C-OPERA) and established RA (Pooled RAPID and EXXELERATE). Additionally, in patients with established RA (EXXELERATE), certolizumab pegol demonstrated consistent efficacy across baseline RF quartiles through Week 104 (data not shown).

In patients with established RA (EXXELERATE), the proportion of patients with high baseline RF levels (Q4) who achieved DAS28 (ESR) LDA or REM at Week 24 was approximately 10 percentage points higher in the certolizumab pegol+MTX group (45.0%) compared to the ADA+MTX group (35.1%). Although inferential statistics were not performed in this analysis, when compared to the other RF quartiles, a slightly lower but still comparable proportion of patients with high baseline RF levels (Q4) responded to certolizumab pegol+MTX treatment at Week 24 (49.4-60.8% vs 45.0%, respectively). However, only 35.1% of patients with high baseline RF levels (Q4) in the ADA+MTX group responded at Week 24, lower than the 48.3-54.7% observed in the other RF quartiles. This suggests that the effect of high baseline RF levels is not as evident on certolizumab pegol efficacy as it is on ADA efficacy. A similar trend was observed at Week 104 (data not shown; inferential statistics not performed), with approximately 15 percentage points more patients in the highest baseline RF level quartile (Q4) achieving DAS28 (ESR) LDA or REM in the certolizumab pegol+MTX group than the ADA+MTX group. Indeed, a recent study of Japanese RA patients grouped by RF quartiles likewise reported higher efficacy of certolizumab pegol compared with other currently available TNFi (analyzed collectively) in the highest baseline RF level quartile (Q4: 166-7,555 IU/mL).21 At 3, 6, and 12 months after treatment, DAS28 (ESR) improvement from baseline was significantly greater in patients who received certolizumab pegol versus other TNFi in the highest baseline RF level quartile.

Studies on TNFi so far have observed lower efficacy in patients with high RF levels compared with patients with low RF levels. However, certolizumab pegol demonstrated consistent efficacy across all RF groups in the current study, including in the highest RF quartile compared with decreased ADA efficacy in the same quartile. This observation may be related in part to its unique molecular Fc-free structure. TNFα inhibitors which are complete monoclonal antibodies, as well as the IgG1-TNFR2 fusion protein etanercept, include the IgG1-Fc fragment which IgM RF bind to; it is reasonable to speculate that TNFi drug bioavailability is reduced when the resulting immune complexes are cleared. Thus, the higher the RF levels are, the higher the impact on clearance of the TNFα inhibitors which are complete monoclonal antibodies or have an Fc fragment, which may explain the lower efficacy observed in patients with high RF levels versus those with low RF levels. In this context, certolizumab pegol, being Fc-free is cleared at a lower rate even in the presence of high RF levels and remains available to inhibit TNF pro inflammatory actions. Interestingly, RF positivity has been associated with better response to rituximab and tocilizumab, both monoclonal antibodies with IgG1-Fc fragments, in patients with RA. However, for these therapies, the association may relate to RF positivity as a B-cell activation marker rather than its role in drug clearance.

Anti-citrullinated peptide antibodies (ACPA) are also important in the pathophysiology of RA and potentially influence efficacy outcomes in patients with RA. In a post hoc analysis of Japanese patients with RA, a combination of high baseline RF (≥160 IU/ml) and ACPA (≥100 U/ml) levels was associated with low drug (infliximab) levels and reduced clinical responses. Given that patients with higher baseline ACPA levels often also have high baseline RF levels, there could be a confounding effect for any inferences on baseline ACPA levels and TNFi efficacy. In the current study, we classified patients by RF levels only due to the hypothesized effect of RF on the clearance of certolizumab pegol and other TNFi. ACPA targets citrullinated proteins and not IgG11 and is therefore unlikely to be involved in the IgG1-Fc clearance process. Furthermore, RF-positive patients were found to have high levels of RA disease activity regardless of ACPA levels, suggesting that the value of determining baseline ACPA levels may be in RF-negative or low-titer RF positive patients rather than in patients with very high baseline RF levels.

A strength of this study is that patients included were from different populations and with a range of disease duration and severity. However, the relatively low patient numbers in the individual clinical trial subgroups may limit the interpretation of results. Furthermore, this diversity may also represent a limitation due to variations in inclusion and exclusion criteria and study periods. The six clinical studies were conducted over different periods (C-OPERA: 2011-2013, RAPID-1 and RAPID 2:2005-2006, J-RAPID: 2008-2010, RAPID C: 2014-2016, and EXXELERATE 2011-2013) and may have reflected changes in clinical practice. Nevertheless, our findings demonstrate that certolizumab pegol showed consistent efficacy trends across the heterogenous trial populations and study periods.

Other potential limitations of the analysis are that observed case data were presented, taking into account responders who continued until at least Week 24, but excluding non-responders who dropped out of the trials. In this study, RF assays may have differed across local hospitals, although all assays were performed using validated techniques. Given that the effect on efficacy is only evident in patients with very high baseline RF levels, the potential variability of RF assays used most likely has a negligible impact on the inferences drawn in this study. Additionally, only IgM RF levels were assayed in this study, although other RF isotypes have also been associated with response rates in patients with RA. For example, high levels (>100 U/mL) of IgA RF have been found to predict poor response rate to TNFi treatment compared with low (20-100 U/mL) or negative (<20 U/mL) levels. Regardless, the most commonly detected RF subtype in patients with RA is IgM-RF and RF levels measured in clinical diagnostic settings usually refer to IgM-RF. As this was a post-hoc analysis, we have not provided quantitative statistical inferences. This study was not powered for hypothesis testing. Nonetheless, we have discussed the numerical differences and potential clinical importance of such differences. Finally, this post-hoc analysis did not assess the safety of certolizumab pegol specific to baseline RF levels. However, the full safety results of all six clinical studies have been published previously and most adverse events were mild or moderate.

In conclusion, this post-hoc analysis provides evidence to support the clinical benefit of certolizumab pegol treatment in association with MTX in RA irrespective of baseline RF status. Certolizumab pegol efficacy was consistent across RF quartiles, including in patients in the quartile with the highest baseline RF levels who are at the highest risk of disease progression.

### Example 3. Impact of High Levels of Rheumatoid Factors on Treatment Outcomes of Certolizumab Pegol and Adalimumab in Patients with Rheumatoid Arthritis: A Post Hoc Analysis of the Phase 4 EXXELERATE Study

### Study Design and Patients

The phase 4 EXXELERATE study (NCT01500278) was a 104-week, randomized, single-blind (double-blind until Week 12 and investigator blind thereafter), parallel-group, head-to-head, superiority trial of CZP versus ADA in patients with RA. Eligible patients were aged 18 or over with a diagnosis of active RA, defined as Disease Activity Score 28-erythrocyte sedimentation rate (DAS28-ESR)>3.2, ≥4/28 swollen joints, and increased acute phase reactants (high sensitivity C-reactive protein [hsCRP]≥10 mg/L and/or ESR≥28 mm/h) at screening and baseline despite a minimum of 12 weeks of methotrexate (MTX) treatment prior to screening. Patients were biologic disease-modifying anti-rheumatic drug (b-DMARD)-naïve and had prognostic factors for severe progression (positive RF and/or anti-citrullinated protein antibodies [ACPA] result) with at least 28 days of stable MTX treatment.

At baseline, patients were randomized 1:1 to CZP (400 mg at Weeks 0, 2, and 4, then 200 mg every 2 weeks [Q2W]) and methotrexate (+MTX) or ADA 40 mg Q2W+MTX. During the first 12 weeks of the study, patients receiving ADA were also administered placebo injections at Weeks 0, 2, and 4 to maintain blinding during the administration of the loading dose of CZP. At Week 12, patients were classified as responders (by achieving low disease activity [LDA]: DAS28-ESR≤3.2 or DAS28-ESR reduction≥1.2 from baseline) or as non-responders. Patients classified as non-responders to the TNFi to which they were randomized were switched to the other TNFi with no washout period. Patients who switched to ADA received 40 mg ADA Q2W+MTX; patients who were switched to CZP received a loading dose of 400 mg CZP at Weeks 12, 14, and 16, followed by CZP 200 mg Q2W+MTX. RF levels were measured by Roche Tina-Quant®.

### Outcomes

We report the following outcomes in CZP and ADA treated patients to Week 104, stratified by baseline RF quartile across all patients (≤Q3 or >Q3; quartile cut-offs chosen to allow for a reasonable number of patients in each subgroup): drug plasma concentration (Sanquin test; performed centrally), mean DAS28-CRP and the proportion of patients achieving DAS28-CRP low disease activity (LDA); DAS28-CRP LDA number needed to treat (NNT); mean DAS28-ESR and the proportion of patients achieving DAS28-ESR LDA; clinical disease activity index (CDAI) score; simplified disease activity index (SDAI) score; the proportion of patients achieving CDAI LDA (CDAI≤10) and SDAI LDA (SDAI≤11); health assessment questionnaire-disability index (HAQ-DI) score, and the proportion of patients achieving revised Boolean remission.

To assess whether the impact of high RF levels was mediated by anti-drug antibodies, a sensitivity analysis was performed after exclusion of patients with high levels of anti-drug antibodies (>50th percentile; threshold chosen arbitrarily, based on the knowledge that higher titers are associated with a higher prevalence of neutralizing antibodies; measured with Sanquin assays) for DAS28-CRP and DAS28-ESR, and the proportion of patients achieving DAS28-CRP LDA and DAS28-ESR LDA.

As a sensitivity analysis to assess whether outcomes are confounded by factors such as disease activity and to ensure our observations are specific to RF level, we also report DAS28-CRP and DAS28-ESR in CZP- and ADA-treated patients to Week 104, stratified by ACPA quartile (≤Q3 or >Q3; measured with immunoassay, performed centrally).

### Analyses

Analysis for outcomes from Weeks 0 to 12 included all patients with baseline and post-baseline efficacy measurements (Full Analysis Set); analysis for outcomes beyond Week 12 included randomized patients who received at least one dose of study drug after Week 12 and had valid baseline, Week 12 and post-Week 12 efficacy measurements (Week 12 Full Analysis Set). At Week 12, patients who were non-responders to the TNFi they were randomized to at baseline switched to the other TNFi; outcomes were analyzed according to the treatment patients were on at the time of measurement.

The area under the curve (AUC) of CZP and ADA plasma concentrations beyond Week 12 were calculated. The AUC analyses did not include patients who switched treatment at Week 12.

Data were reported as observed case (OC) for all outcomes other than revised Boolean remission (non-responder imputation [NRI]). DAS28-CRP LDA and DAS28-ESR LDA are reported as OC and NRI. Reported p values were not pre-specified and are therefore nominal, and so should be interpreted with caution.

### Results

### Patient Demographics and Baseline Characteristics

Baseline data by RF quartiles were available for 453 CZP-randomized patients (≤Q3 [RF≤204 IU/mL]: n=334; >Q3 [RF>204 IU/mL]: n=119) and 454 ADA-randomized patients (≤204 IU/mL: n=347; >204 IU/mL n=107). At Week 12, 66 CZP-randomized patients switched to ADA and 59 ADA-randomized patients switched to CZP. Baseline demographics and characteristics were similar between ADA- and CZP-randomized groups with RF≤204 IU/mL and >204 IU/mL, though patients with high RF levels had greater mean disease duration (Table 1).

TABLE 1
Baseline demographics and patient characteristics stratified by RF

| | ≤Q3 (RF ≤204 IU/mL) | | | | >Q3 (RF >204 IU/mL) | | | |
|---|---|---|---|---|---|---|---|---|
| | CZP + MTX | | ADA + MTX | | CZP + MTX | | ADA + MTX | |
| | (N = 334) | | (N = 347) | | (N = 119) | | (N = 107) | |
| Age (years), mean (SD) | 52.7 | (12.4) | 52.5 | (12.9) | 55.5 | (11.7) | 54.2 | (12.3) |
| BMI (kg/m²), mean (SD) | 28.37 | (6.35) | 28.09 | (6.02) | 28.77 | (6.29) | 27.38 | (7.04) |
| Sex (male, %) | 19.2 | | 20.5 | | 26.9 | | 22.4 | |
| Baseline steroid use (yes, %) | 47.9 | | 55.9 | | 68.1 | | 60.7 | |
| Previous TNFi use (yes, %) | 0.6 | | 0.3 | | 0.0 | | 0.9 | |
| Disease duration (years), mean | 5.54 | (6.20) | 5.45 | (6.50) | 7.54 | (8.58) | 6.94 | (7.84) |
| (SD) | | | | | | | | |
| Baseline DAS28-CRP | | | | | | | | |
| n | 332 | | 346 | | 118 | | 106 | |
| mean (SD) | 5.52 | (0.87) | 5.65 | (0.85) | 5.89 | (0.91) | 5.91 | (0.96) |
| Baseline DAS28-ESR | | | | | | | | |
| n | 334 | | 346 | | 118 | | 107 | |
| mean (SD) | 6.40 | (0.86) | 6.49 | (0.82) | 6.72 | (0.90) | 6.70 | (0.93) |
| MTX dose (mg/week), mean (SD) | 17.5 | (3.6) | 18.1 | (3.9) | 17.8 | (4.3) | 17.9 | (4.0) |

Full Analysis Set. ADA: adalimumab; BMI: body mass index; CRP: C-reactive protein; CZP: certolizumab pegol; DAS28: Disease Activity Score-28 joint count; ESR: erythrocyte sedimentation rate; MTX: methotrexate; Q3: third quartile; RF: rheumatoid factor; SD: standard deviation; TNFi: tumor necrosis factor inhibitor.

### Drug Concentrations Stratified by RF Level

FIG. 5B illustrates the drug plasma concentrations of CZP 200 mg and ADA 40 mg to Week 104. From Week 12 onwards, the AUC of ADA drug concentration was lower in patients with RF levels>204 IU/mL than in patients with RF levels≤204 IU/mL; the AUC of CZP concentration was similar in patients with RF≤204 IU/mL and >204 IU/mL.

### Efficacy Stratified by RF Level

Clinical outcomes were poorer in patients with RF levels>204 IU/mL who were treated with ADA compared to those treated with CZP. For patients with RF≤204 IU/mL, mean (SD) DAS28-CRP was similar between CZP- and ADA-treated patients at Week 104 (CZP: 2.5 [1.2]; ADA: 2.5 [1.1]; nominal p=0.917; FIG. 3b). However, for patients with RF>204 IU/mL, mean (SD) DAS28-CRP was lower in CZP-versus ADA-treated patients at Week 104 (CZP: 2.5 [1.2]; ADA: 2.9 [1.2]; nominal p=0.046; FIG. 3b). The proportion of patients with RF≤204 IU/mL achieving DAS28-CRP LDA was similar between CZP-(127/196; 64.8%) and ADA-treated (151/232; 65.1%) patients at Week 104 (OC data; FIG. 3b). However, for patients with RF>204 IU/mL, the proportion achieving DAS28-CRP LDA was higher (36% relative increase; OC data) at Week 104 in CZP-(46/70; 65.7%) versus ADA-(28/58; 48.3%) treated patients. Findings were similar when NRI was utilized (FIG. 3b). NNT at Week 104 for DAS28-CRP LDA in patients with RF≤204 IU/mL was 52.1 for CZP versus ADA (95% confidence interval: 193.5-297.6) and 5.5 (95% confidence interval: 0.3-10.7) in patients with RF>204 IU/mL. A similar pattern was observed for DAS28-ESR and DAS28-ESR LDA (FIG. 8D)

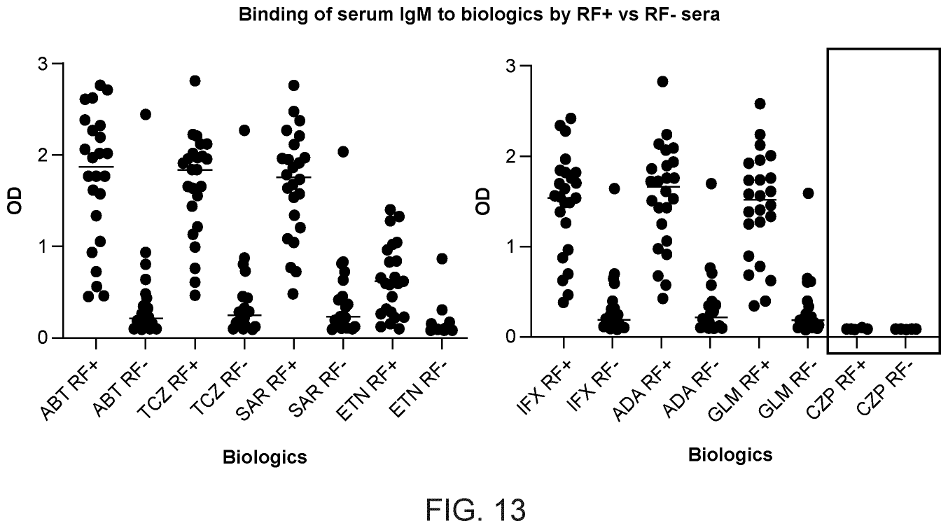

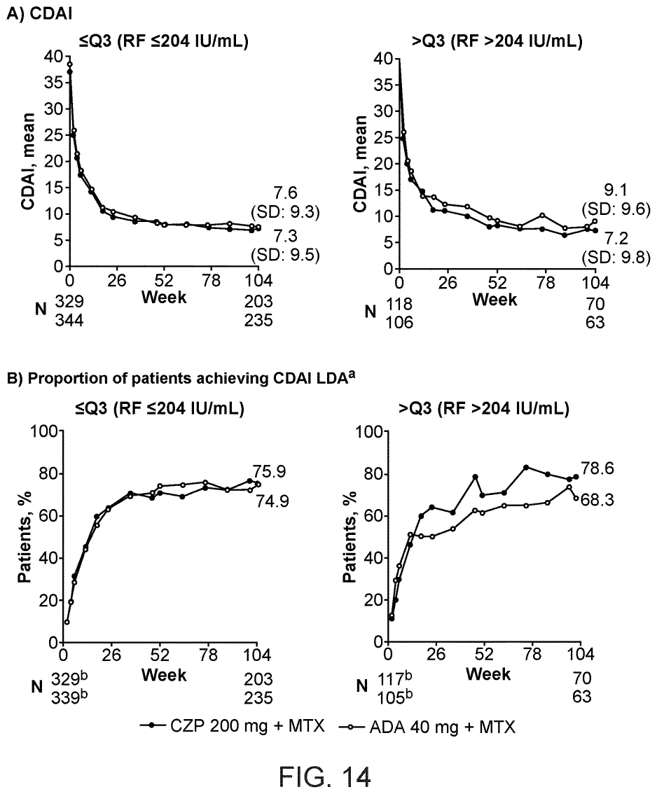

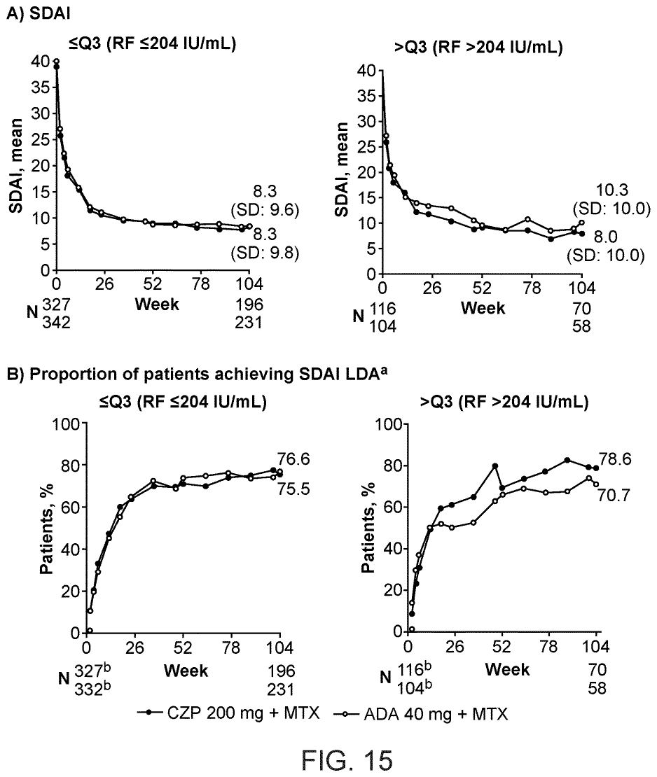

Response to CZP and ADA, as measured by CDAI and CDAI LDA are illustrated in FIG. 13. SDAI and SDAI LDA are available in Supplementary FIG. 14. At Week 104, mean (SD) CDAI was similar in CZP- and ADA-treated patients with RF levels≤204 IU/mL (CZP: 7.3 [9.5]; ADA: 7.6 [9.3]). In patients with RF levels>204 IU/mL, mean (SD) CDAI was numerically lower in CZP-versus ADA-treated patients (CZP: 7.2 [9.8]; ADA: 9.1 [9.6]). The proportion of patients achieving CDAI LDA was similar for patients with RF levels≤204 IU/mL at Week 104 (CZP: 75.9%; ADA: 74.9%) while in patients with RF>204 IU/mL, the proportion of patients achieving CDAI LDA was higher in CZP-versus ADA-treated patients (CZP: 78.6%; ADA: 68.3%; FIG. 13). A similar pattern was observed for SDAI and SDAI LDA (FIG. 14). HAQ-DI scores were similar between CZP- and ADA-treated patients with RF level≤204 IU/mL and >204 IU/mL at Week 104 (FIG. 15).

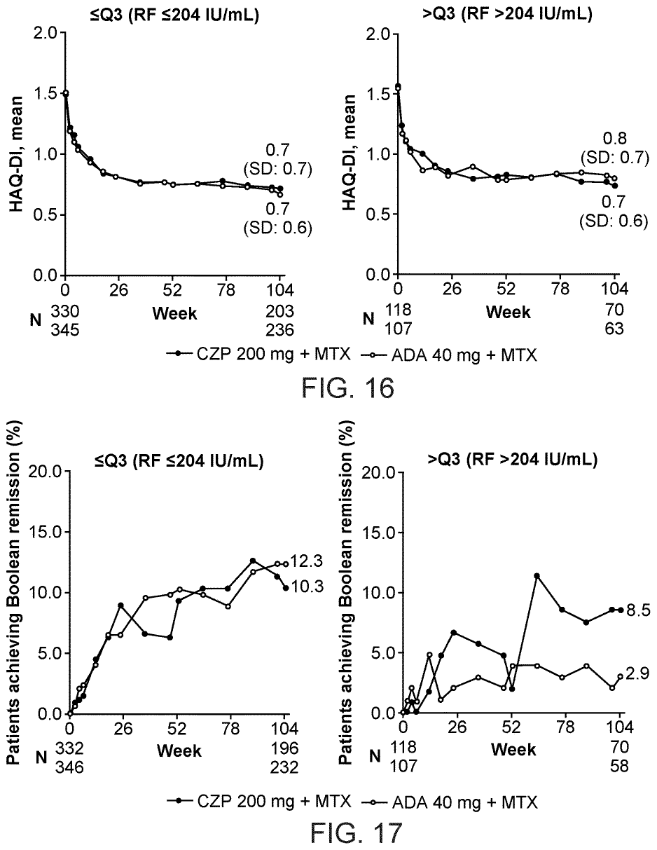

Treatment response measured by revised Boolean remission is illustrated in FIG. 16; from Week 64, in patients with RF level>204 IU/mL, a higher proportion of CZP-treated patients achieved Boolean remission compared with ADA-treated patients (CZP: 8.5% at Week 104; ADA: 2.9% at Week 104).

Sensitivity Analyses: Exclusion of Patients with High Levels of Anti-Drug Antibodies

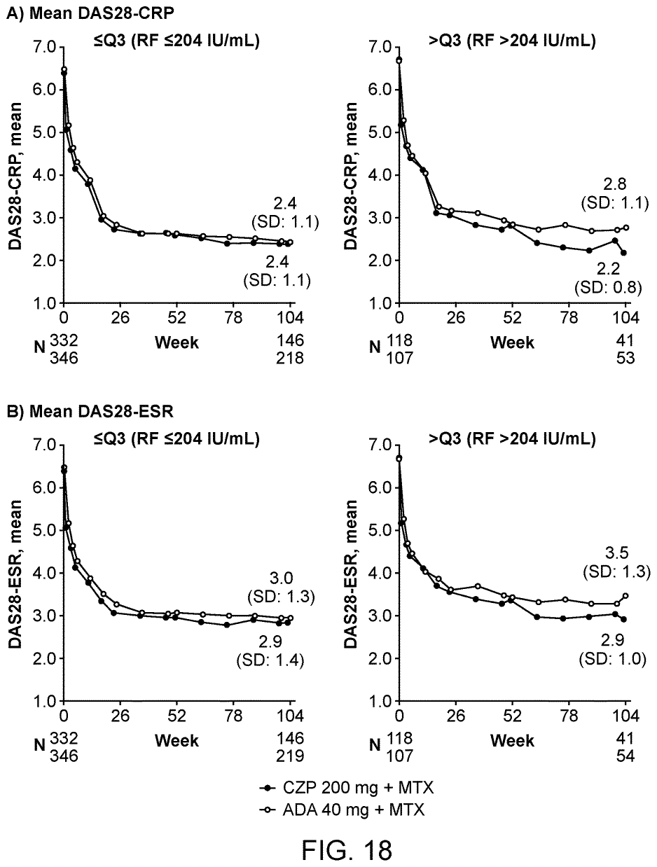

To assess whether the impact of high RF levels on treatment with ADA and CZP was mediated by anti-drug antibodies, DAS28-CRP, DAS28-ESR, DAS28-CRP LDA, and DAS28-ESR LDA after exclusion of patients with high levels of anti-drug antibodies (>50th percentile) were analyzed. DAS28-CRP, DAS28-ESR, DAS28-CRP LDA, and DAS28-ESR LDA after exclusion of patients with high levels of anti-drug antibodies (>50th percentile) are illustrated in FIGS. 17 and 18 and are consistent with findings from the whole study population. For patients with RF levels≤204 IU/mL, mean (SD) DAS28-CRP and DAS28-ESR were similar between CZP- and ADA-treated patients at Week 104 (DAS28-CRP: 2.4 [1.1] CZP and 2.4 [1.1] ADA; DAS28-ESR: 2.9 [1.4] CZP and 3.0 [1.3] ADA) after exclusion of patients with high levels of anti-drug antibodies (FIG. 17). However, for patients with RF levels>204 IU/mL, mean DAS28-CRP and DAS28-ESR were lower in CZP-treated patients compared with ADA-treated patients at Week 104 (DAS28-CRP: 2.2 [0.8] CZP and 2.8 [1.1] ADA; DAS28-ESR: 2.9 [1.0] CZP and 3.5 [1.3] ADA) after exclusion of patients with high levels of anti-drug antibodies.

The proportions of patients achieving DAS28-CRP LDA and DAS28-ESR LDA after exclusion of patients with high levels of anti-drug antibodies were also similar in patients with RF levels≤204 IU/mL at Week 104 (67.8% CZP and 66.5% ADA; DAS28-ESR: 67.1% CZP and 62.1% ADA; FIG. 18). There was differentiation, however, between patients with RF levels>204 IU/mL at Week 104 (DAS28-CRP LDA: 78.0% CZP and 52.8% ADA; DAS28-ESR: 65.9% CZP and 44.4% ADA).

### Sensitivity Analyses: Efficacy Stratified by ACPA Level

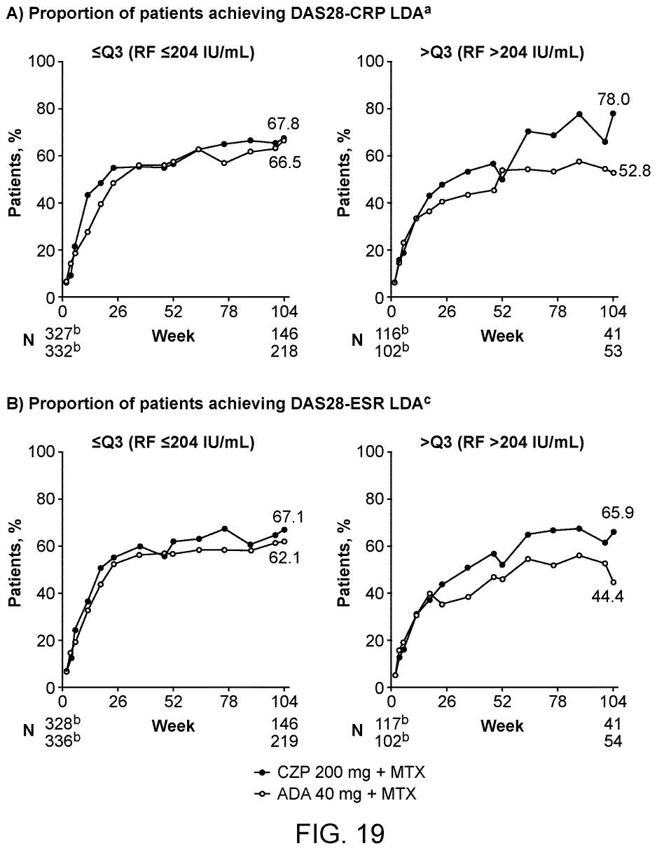

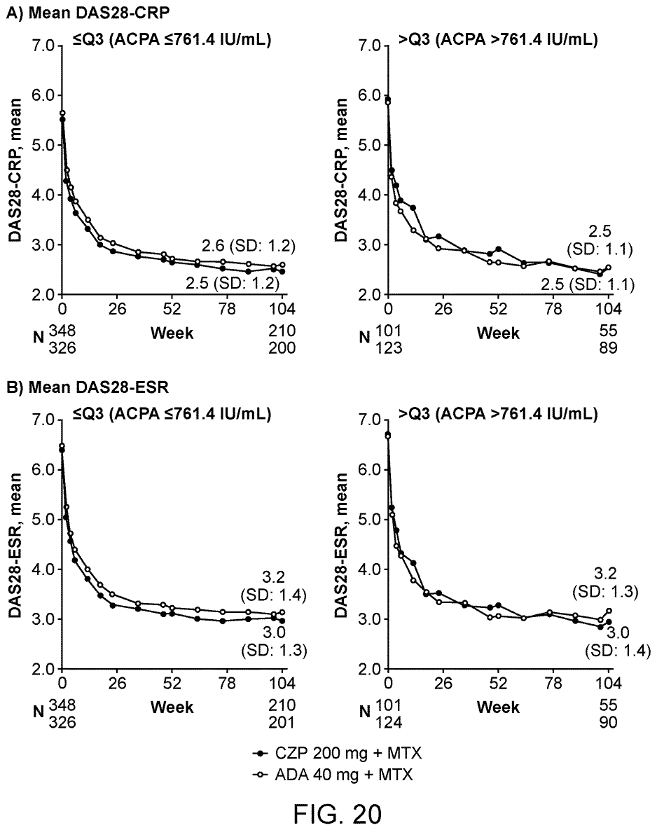

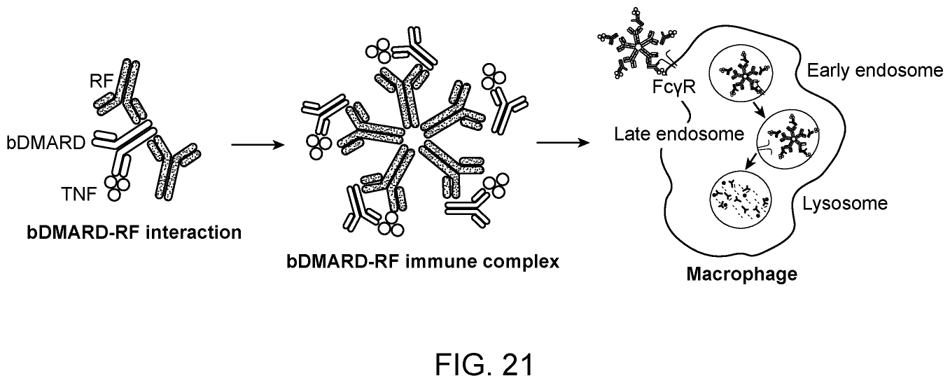

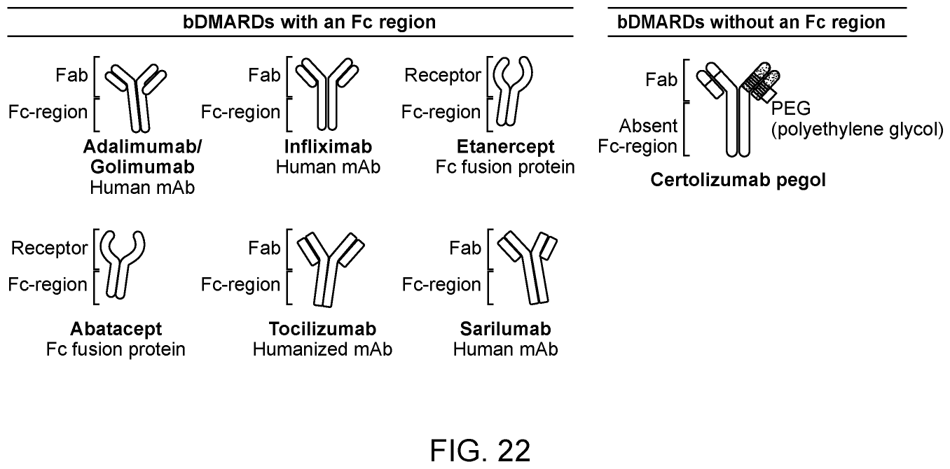

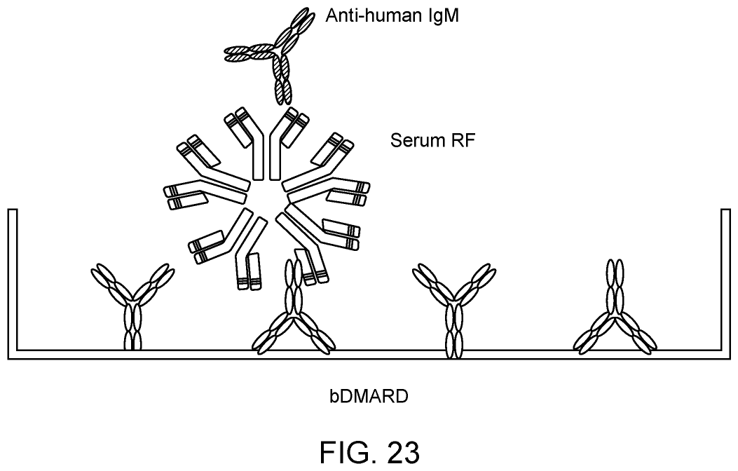

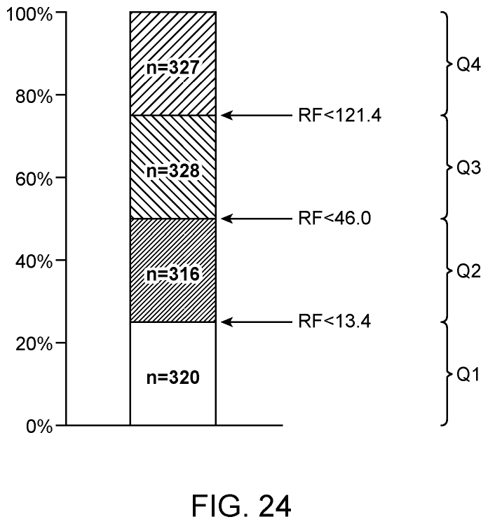

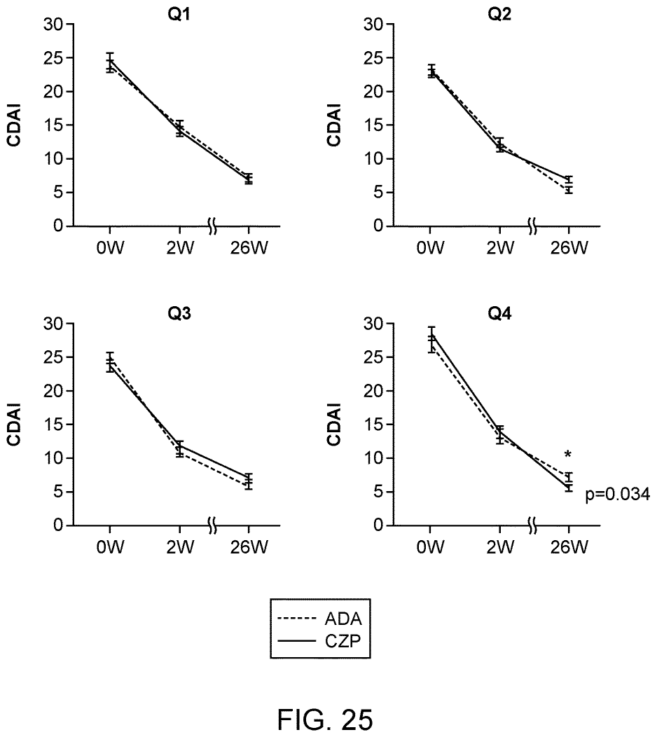

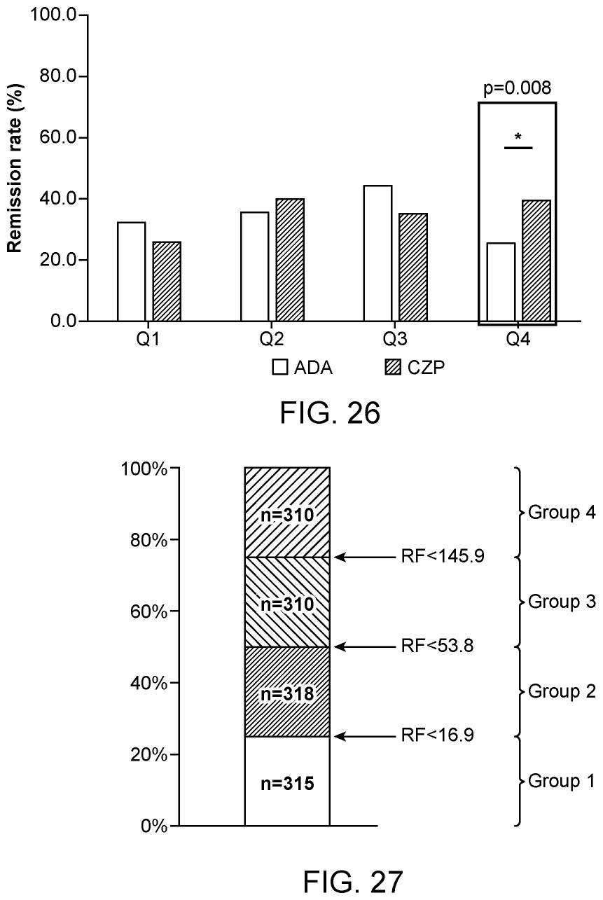

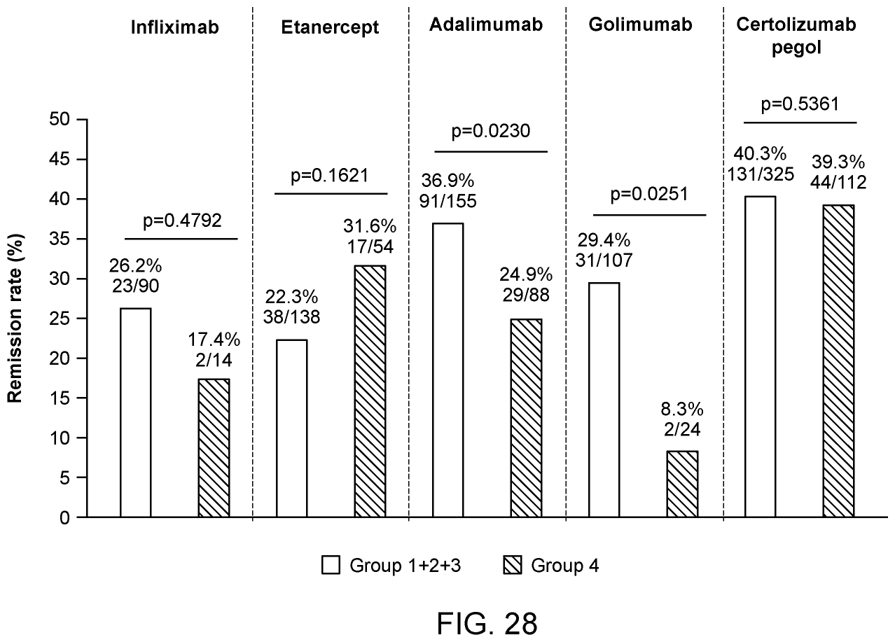

DAS28-CRP and DAS28-ESR were similar through Weeks 0-104 between CZP- and ADA-treated patients with both ACPA levels≤761.4 IU/mL and >761.4 IU/mL (FIG. 19). At Week 104, in patients with ACPA levels>761.4 IU/mL, mean (SD) DAS29-CRP was 2.5 (1.1) in CZP-treated patients and 2.5 (1.1) in ADA-treated patients and; mean (SD) DAS28-ESR was 3.2 (1.3) in ADA-treated patients and 3.0 (1.4) in CZP-treated patients. There was poor correlation between RF and ACPA levels (data not shown).

### Discussion

This post hoc analysis of the EXXELERATE study provides a direct comparison of CZP versus ADA in patients with RA and high RF levels. In patients with RF levels in the highest quartile, clinical outcomes were more favorable in those who were treated with CZP compared with patients who received ADA. Further, drug concentrations were lower in patients with high RF levels compared to lower RF levels in ADA-treated patients, while such a difference was not observed in CZP-treated patients. The results of sensitivity analyses to assess the effects of anti-drug antibodies and ACPA levels indicate that these findings were not confounded by factors such as the presence of anti-drug antibodies or diseases activity.

In a previous analysis of the effect of ADA plasma concentration on treatment response among patients with high RF levels, higher ADA levels were associated with better response and improved drug survival. In that study, a lower therapeutic threshold of 6.0 mg/L was suggested; in this post hoc analysis, plasma ADA levels reached 4.8 μg/mL at Week 104 in patients with RF>204 IU/mL, which indicates that the lower serum drug concentration in ADA-treated patients with high levels of RF may have led to the lower efficacy compared with CZP. On the other hand, CZP-treated patients with RA and high RF levels had similar drug concentrations and maintained clinical outcomes compared with those with lower RF levels.

In patients with RA and RF levels>204 IU/mL, the proportion of patients achieving DAS28-CRP LDA and DAS28-ESR LDA at Week 104 were 36% and 38% higher in CZP-treated patients relative to ADA-treated patients, respectively; there was little difference between CZP- and ADA-treated patients with RF levels≤204 IU/mL. The response to CZP and ADA, as measured by CDAI and SDAI, was also greater in patients with RA treated with CZP compared with ADA in patients with RF levels>204 IU/mL, but not in those with RF levels≤204 IU/mL.

In this analysis, the differences in CDAI observed between CZP- and ADA-treated patients with high RF levels were clinically important. The minimal clinically important difference cut-off for CDAI has previously been defined as 1 when CDAI is <10. Here, in patients with RF levels>204 IU/mL, CDAI was 1.9 points lower in CZP-treated patients compared with ADA-treated patients at Week 104. Furthermore, NNT at Week 104 for DAS28-CRP LDA was approximately 10-times greater in patients with RF levels≤204 IU/mL than in patients with RF levels>204 IU/mL (≤204 IU/mL: 52.1; >204 IU/mL: 5.5), indicating that in patients with high RF levels, there was greater clinical benefit of CZP compared to ADA than in patients with low RF levels. We found that DAS28-CRP and DAS28-ESR were similar in CZP- and ADA-treated patients with high and low levels of ACPA. The absence of an impact of high levels of ACPA on response to CZP and ADA indicates that our observations were not confounded by ACPA levels as an autoantibody characteristic of RA that does not bind to the Fc-portion of IgG, hence suggesting a true biologic effect of RF.

We also found similar efficacy results when patients with high concentrations of anti-drug antibodies were excluded from the analysis. This suggests that anti-drug antibodies do not contribute to the difference observed in clinical outcomes between patients with RA and high RF levels when treated with either CZP or ADA.

These findings are consistent with previous reports from indirect comparisons that suggested consistent efficacy of CZP irrespective of baseline RF level, while patients with high RF levels who received Fc-containing TNF is appeared to have lower drug concentrations and response to therapy. Of note, data from the current analysis have been derived from a randomized controlled trial which directly compared CZP with ADA.

### Conclusion

Patients with RA and high levels of RF are a subgroup of patients with poor clinical outcomes. In ADA-treated patients, clinical outcomes were poorer and drug concentration lower in patients with high RF levels compared with lower RF levels. However, in CZP-treated patients, those with high levels of RF had similar drug concentration and clinical outcomes to patients with lower levels of RF. Thus, the current data are the first from a head-to-head trial comparing CZP and ADA to show that response to therapy in patients with RA and high levels of RF is influenced by the presence or absence of an Fc-portion, which is in line with previous findings.

### Example 4

Assessment of efficacy outcomes in patients with RA treated with certolizumab pegol (CZP) (a Fc-free TNFi) or adalimumab (ADA) (an Fc-containing TNFi), stratified by RF level.

Clinical Data: Study Design in a retrospective analysis of FIRST Registry in a Japanese population split by RF quartiles:

FIRST registry (n=5,077): Patients with RA who started molecular-targeted antirheumatic drug therapy. Patients who started ADA or CZP were followed up for ≥1 year between January 2012^{a}-May 2022 (n=1,253). [a] Year in which our hospital complied with the JCR's reference to the standardization of RF measurements.

Patients were stratified by RF level according to the following groups:
Group 1: RF<13.4 (n=163),
Group 2:13.4≤RF<46.0 (n=164),
Group 3:46.0≤RF<121.4 (n=164), and
Group 4:121.4≤RF (n=164).

TABLE 2
Baseline Characteristics (Pre PS-ITPW) shows the clinical data baseline
characteristics. The table includes patients in quartiles Q1 and Q2
before propensity score-matching (statistical method to compare only
similar patients as per specific variables to avoid potential biases).

| | Q1 | | | Q2 | | |
|---|---|---|---|---|---|---|
| | ADA | CZP | p | ADA | CZP | p |
| | n = 72 | n = 91 | value | n = 68 | n = 96 | value |
| Female, n (%) | 55 | 73 | 0.554 | 59 | 82 | 0.807 |
| | (72%) | (18%) | | (86.8%) | (85.4%) | |
| Age, years | 54.3 | 58.4 | 0.051 | 54.6 | 56.8 | 0.208 |
| | (12.6) | (14.4) | | (13.5) | (14.3) | |
| Disease duration, | 43.2 | 49.0 | 0.474 | 64.4 | 54.0 | 0.143 |
| months | (51.5) | (56.7) | | (86.3) | (85.6) | |
| Stage I/II/III/IV, n | 37/31/3/1 | 4/43/4/0 | 0.682 | 22/33/7/6 | 39/50/6/1 | 0.063 |
| Methotrexate dose, | 13.3 | 11.9 | 0.010 | 13.9 | 12.7 | 0.052 |
| mg/w | (3.1) | (3.5) | | (3.2) | (3.4) | |
| Prednisolone dose, | 0.1 | 0.2 | 0.762 | 0.3 | 0.2 | 0.530 |
| mg/day | (0.6) | (0.8) | | (1.0) | (0.8) | |
| Prior bDMARDs, | 55/14/3/0 | 62/26/3/0 | 0.401 | 48/18/2/0 | 79/8/9/0 | 0.003 |
| 0/1/2/3/≥4, n | | | | | | |
| Clinical Disease | 22.6 | 27.1 | 0.185 | 21.7 | 23.8 | 0.638 |
| Activity Index | (10.7) | (16.3) | | (8.6) | (12.6) | |
| HAQ-DI | 0.852 | 1.080 | 0.118 | 0.853 | 0.918 | 0.641 |
| | (0.695) | (0.841) | | (0.666) | (0.732) | |
| C-reactive protein, | 1.18 | 1.50 | 0.468 | 1.06 | 1.34 | 0.877 |
| mg/dL | (2.69) | (2.86) | | (1.58) | (2.72) | |
| ESR, mm/h | 28 | 34 | 0.094 | 34 | 35 | 0.848 |
| | (25) | (27) | | (25) | (26) | |
| Rheumatoid factor, | 5.7 | 5.2 | 0.367 | 26.1 | 27.2 | 0.285 |
| U/mL | (3.4) | (4.2) | | (9.9) | (9) | |

TABLE 3
Baseline Characteristics (Pre PS-ITPW) shows the clinical data baseline
characteristics. The table includes patients in quartiles Q3 and Q4
before propensity score-matching (statistical method to compare only
similar patients as per specific variables to avoid potential biases).

| | Q3 | | | Q4 | | |
|---|---|---|---|---|---|---|
| | ADA | CZP | p | ADA | CZP | p |
| | n = 63 | n = 101 | value | n = 54 | n = 110 | value |
| Female, n (%) | 53 | 85 | 0.996 | 42 | 82 | 0.651 |
| | (84.1%) | (84.2%) | | (77.8%) | (74.6%) | |
| Age, years | 57 | 56.5 | 0.772 | 55.3 | 57 | 0.382 |
| | (13.5) | (13.9) | | (13.9) | (14.7) | |
| Disease duration, | 86.5 | 83.3 | 0.583 | 84.4 | 92.9 | 0.700 |
| months | (121.1) | (99.1) | | (98.6) | (111.6) | |
| Stage I/II/III/IV, n | 19/27/12/5 | 29/50/13/9 | 0.704 | 17/25/6/6 | 38/42/19/11 | 0.653 |
| Methotrexate dose, | 12.6 | 13 | 0.300 | 12.7 | 12 | 0.143 |
| mg/w | (3.2) | (3.3) | | (3.9) | (3.5) | |
| Prednisolone dose, | 0.2 | 0.2 | 0.901 | 0.5 | 0.3 | 0.119 |
| mg/day | (0.8) | (0.9) | | (1.3) | (1.1) | |
| Prior bDMARDs, | 43/15/5/0 | 73/22/5/1 | 0.717 | 37/14/2/1 | 72/25/7/6 | 0.620 |
| 0/1/2/3/≥4, n | | | | | | |
| Clinical Disease | 23.5 | 24.6 | 0.709 | 30.0 | 27.7 | 0.389 |
| Activity Index | (11.7) | (12.9) | | (15.2) | (12.9) | |
| HAQ-DI | 1.063 | 1.105 | 0.591 | 1.236 | 1.116 | 0.478 |
| | (0.758) | (0.722) | | (0.871) | (0.773) | |
| C-reactive protein, | 1.53 | 2.04 | 0.092 | 2.14 | 2.06 | 0.384 |
| mg/dL | (2.91) | (3.08) | | (2.63) | (2.84) | |
| ESR, mm/h | 39 | 45 | 0.147 | 53 | 50 | 0.666 |
| | (28) | (27) | | (32) | (31) | |
| Rheumatoid factor, | 75.2 | 77.1 | 0.729 | 397 | 343.3 | 0.485 |
| U/mL | (19.3) | (21.9) | | (467.7) | (342.5) | |

TABLE 4
Characteristics (Post PS-ITPW) shows the clinical data baseline characteristics.
The table includes patients in quartiles Q1 and Q2 after propensity
score-matching (statistical method to compare only similar patients
as per specific variables to avoid potential biases).

| | Q1 | | | Q2 | | |
|---|---|---|---|---|---|---|
| | ADA | CZP | p | ADA | CZP | p |
| | n = 157 | n = 163 | value | n = 156 | n = 160 | value |
| Female, n (%) | 122 | 128 | 0.859 | 141 | 138 | 0.316 |
| | (77.7%) | (78.5%) | | (90.4%) | (86.8%) | |
| Age, years | 56 | 56.4 | 0.829 | 53.6 | 56.2 | 0.103 |
| | (1.0) | (1.2) | | (1.1) | (1.1) | |
| Disease duration, | 46.2 | 46 | 0.968 | 54.0 | 51.8 | 0.799 |
| months | (4.3) | (4.1) | | (6.0) | (6.5) | |
| Stage I/II/III/IV, n | 76/74/6/1 | 77/79/7/0 | 0.769 | 54/83/12/7 | 60/85/12/3 | 0.596 |
| Methotrexate dose, | 12.8 | 12.6 | 0.488 | 13.7 | 13.1 | 0.127 |
| mg/w | (0.3) | (0.3) | | (0.2) | (0.3) | |
| Prednisolone dose, | 0.1 | 0.2 | 0.714 | 0.2 | 0.2 | 0.703 |
| mg/day | (0.0) | (0.1) | | (0.1) | (0.1) | |
| Prior bDMARDs, | 115/37/6/0 | 117/41/5/0 | 0.889 | 118/26/12/0 | 127/20/12/0 | 0.581 |
| 0/1/2/3/≥4, n | | | | | | |
| Clinical Disease | 23.9 | 24.7 | 0.586 | 23.2 | 23.1 | 0.911 |
| Activity Index | (0.9) | (1.2) | | (0.7) | (0.9) | |
| HAQ-DI | 0.917 | 0.988 | 0.401 | 0.892 | 0.886 | 0.938 |
| | (0.058) | (0.062) | | (0.051) | (0.057) | |
| C-reactive protein, | 1.31 | 1.33 | 0.929 | 1.20 | 1.21 | 0.959 |
| mg/dL | (0.23) | (0.21) | | (0.14) | (0.21) | |
| ESR, mm/h | 30 | 31 | 0.737 | 36 | 35 | 0.557 |
| | (2) | (2) | | (2) | (2) | |
| Rheumatoid factor, | 5.3 | 5.3 | 1.000 | 27.0 | 26.6 | 0.697 |
| U/mL | (0.3) | (0.3) | | (0.8) | (0.7) | |

TABLE 5
Characteristics (Post PS-ITPW). The table includes patients in quartiles Q3
and Q4 after propensity score-matching (statistical method to compare only
similar patients as per specific variables to avoid potential biases).

| | Q3 | | | Q4 | | |
|---|---|---|---|---|---|---|
| | ADA | CZP | p | ADA | CZP | p |
| | n = 165 | n = 163 | value | n = 163 | n = 164 | value |
| Female, n (%) | 141 | 138 | 0.841 | 120 | 122 | 0.874 |
| | (85.5%) | (84.7%) | | (73.6%) | (74.4%) | |
| Age, years | 56.1 | 56.5 | 0.800 | 56.8 | 56.4 | 0.795 |
| | (1.1) | (1.1) | | (1.0) | (1.1) | |
| Disease duration, | 88.0 | 85.9 | 0.867 | 89.8 | 90.5 | 0.950 |
| months | (10) | (7.8) | | (7.5) | (8.8) | |
| Stage I/II/III/IV, n | 54/83/12/7 | 60/85/12/3 | 0.596 | 51/69/28/16 | 55/66/25/17 | 0.937 |
| Methotrexate dose, | 13.0 | 12.9 | 0.854 | 12.3 | 12.2 | 0.821 |
| mg/w | (0.2) | (0.3) | | (0.3) | (0.3) | |
| Prednisolone dose, | 0.2 | 0.2 | 1.000 | 0.4 | 0.4 | 0.852 |
| mg/day | (0.1) | (0.1) | | (0.1) | (0.1) | |
| Prior bDMARDs, | 119/37/8/0 | 116/37/8/1 | 0.993 | 105/39/10/9 | 109/39/9/7 | 0.946 |
| 0/1/2/3/≥4, n | | | | | | |
| Clinical Disease | 25.0 | 23.9 | 0.400 | 26.6 | 28.2 | 0.299 |
| Activity Index | (0.9) | (1) | | (1.2) | (1.0) | |
| HAQ-DI | 1.063 | 1.071 | 0.927 | 1.108 | 1.171 | 0.482 |
| | (0.06) | (0.056) | | (0.065) | (0.061) | |
| C-reactive protein, | 1.78 | 1.82 | 0.900 | 1.94 | 2.06 | 0.676 |
| mg/dL | (0.25) | (0.23) | | (0.20) | (0.22) | |
| ESR, mm/h | 41 | 42 | 0.575 | 53 | 51 | 0.633 |
| | (2) | (2) | | (2) | (2) | |
| Rheumatoid factor, | 74.1 | 75.8 | 0.464 | 351.5 | 361.0 | 0.825 |
| U/mL | (1.5) | (1.7) | | (31.8) | (28.8) | |

Assessment efficacy outcomes in patients with RA treated with CZP (Fc-free TNFi) or ADA (Fc-containing TNFi), stratified by RF level.

FIRST Registry Study Design of a retrospective analysis of the Japanese FIRST Registry comparing patients treated beyond 1 year with CZP or ADA stratified by baseline RF quartiles. To limit biased during the statistical analysis only patients with similar characteristics (adjusting by confounding factors) were included in the comparison.

FIRST registry (n=5,077): Patients with RA who started molecular-targeted antirheumatic drug therapy. Patients who started ADA or CZP were followed up for ≥1 year between January 2012^{a}-May 2022 (n=1,253). [a] Year in which our hospital complied with the JCR's reference to the standardization of RF measurements.

Patients were stratified by RF level according to the following groups:
Group 1: RF<16.9 (n=315),
Group 2:16.9≤RF<53.8 (n=318),
Group 3:53.8≤RF<145.9 (n=310), and
Group 4:145.9≤RF (n=310).

TABLE 6
FIRST Registry Baseline Characteristics. The table shows the demographic baseline characteristics of
patients under different bDMARDs treatment obtained from FIRST Registry. All these patients were used
in the experiment to demonstrate potential binding of RF to the respective Fc fragments. The group
of patients treated with ADA and CZP were the set of subjects used in the ADA vs CZP comparison.

| | | | | | Certolizumab | |
|---|---|---|---|---|---|---|
| | Infliximab | Etanercept | Adalimumab | Golimumab | pegol | p |
| | (n = 113) | (n = 192) | (n = 353) | (n = 151) | (n = 444) | value |
| Age, years, | 57.0 ± 14.0 | 68.9 ± 12.6 | 55.7 ± 13.4 | 66.0 ± 13.2 | 56.2 ± 14.5 | <0.0001 |
| mean (SD) | | | | | | |
| Female, n (%) | 93 | 154 | 287 | 122 | 355 | 0.9776 |
| | (82.3%) | (80.2%) | (81.3%) | (80.8%) | (80.0%) | |
| Disease duration, | 18 | 59 | 26 | 118 | 36 | <0.0001 |
| months, median | (8-72) | (17-180) | (10-92) | (48-204) | (10-103) | |
| (IQR) | | | | | | |
| Stage | 34/60/12/7 | 34/76/47/35 | 132/160/39/22 | 28/46/46/31 | 165/205/48/26 | <0.0001 |
| I/II/III/IV, n | | | | | | |
| Methotrexate | 11.2 ± 4.0 | 11.2 ± 4.0 | 11.2 ± 4.0 | 11.2 ± 4.0 | 11.2 ± 4.0 | — |
| dose, mg/w, | | | | | | |
| mean (SD) | | | | | | |
| Prednisolone | 8.9 ± 11.6 | 8.9 ± 11.6 | 8.9 ± 11.6 | 8.9 ± 11.6 | 8.9 ± 11.6 | — |
| dose, mg/day, | | | | | | |
| mean (SD) | | | | | | |
| Prior | 95/12/2/4/1 | 131/47/10/2/2 | 259/79/14/0/1 | 30/62/30/17/12 | 306/93/27/10/8 | <0.0001 |
| bDMARDS, | | | | | | |
| 0/1/2/3/≥4, n | | | | | | |
| CDAI, mean | 24.5 ± 12.6 | 26.5 ± 12.1 | 23.5 ± 11.8 | 23.8 ± 11.9 | 26.3 ± 14.0 | 0.0095 |
| (SD) | | | | | | |
| HAQ-DI, | 1.2 ± 0.8 | 1.7 ± 0.9 | 1.0 ± 0.8 | 1.4 ± 0.8 | 1.1 ± 0.8 | <0.0001 |
| mean (SD) | | | | | | |
| C-reactive | 0.5 | 1.6 | 0.4 | 0.6 | 0.5 | <0.0001 |
| protein, mg/dL, | (0.1-1.6) | (0.4-4.6) | (0.1-1.7) | (0.1-2.0) | (0.1-2.1) | |
| median (IQR) | | | | | | |
| ESR, mm/h, | 34 | 62 | 31 | 42 | 34 | <0.0001 |
| median (IQR) | (18-62) | (33-82) | (14-58) | (23-64) | (18-64) | |
| MMP-3, ng/mL, | 98.0 | 176.5 | 85.0 | 127.0 | 100.0 | <0.0001 |
| median (IQR) | (53.0-215.5) | (80.5-328.5) | (49.2-188.5) | (59.1-253.5) | (50.8-236.3) | |
| RF, U/mL, | 45.2 | 81.0 | 40.4 | 75.1 | 54.4 | <0.0001 |
| median (IQR) | (14.5-126.7) | (23.0-193.0) | (13.0-122.4) | (22.7-205.4) | (16.4-139.6) | |
| Anti-CCP | 84 | 147 | 239 | 120 | 316 | 0.0375 |
| antibody | (74.3%) | (77.4%) | (67.9%) | (79.5%) | (71.3%) | |
| positive, n (%) | | | | | | |

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method of treating a rheumatic disease in a human patient having rheumatoid factor comprising:
administering a bDMARD lacking an Fc fragment to a human patient identified as having rheumatoid factor above 100 IU/ml, above 121.4 IU/ml, above 125 IU/ml, above 145.9 IU/ml, above 150 IU/ml, above 175 IU/ml, above 200 IU/ml or above 204 IU/ml in serum;
wherein (i) the therapeutic effect of the bDMARD lacking an Fc fragment is not reduced by RF in said patient and/or (i) the treatment induces low disease activity or achieves substantial clinical benefit in the patient.

2. A method of treating a rheumatic disease in a human patient having rheumatoid factor comprising:
a) determining the titer of rheumatoid factor in a sample, preferably a serum sample, from said patient;
b) administering a bDMARD lacking an Fc fragment to said patient if the titer of rheumatoid factor is above 100 IU/ml, above 121.4 IU/ml, above 125 IU/ml, above 145.9 IU/ml, above 150 IU/ml, above 175 IU/ml, above 200 IU/ml, above 204 IU/ml, above 225 IU/ml, above 250 IU/ml, or above 275 IU/ml in serum;
wherein (i) the therapeutic effect of the bDMARD lacking an Fc fragment is not reduced by RF in said patient and/or (i) the treatment induces low disease activity or achieves substantial clinical benefit in the patient.

3. A method of treating rheumatoid arthritis in a human patient having rheumatoid factor comprising:
administering certolizumab pegol to a human patient identified as having rheumatoid factor above 100 IU/ml, above 121.4 IU/ml, above 125 IU/ml, above 145.9 IU/ml, above 150 IU/ml, above 175 IU/ml, above 200 IU/ml or above 204 IU/ml in serum;
wherein (i) the therapeutic effect of certolizumab pegol is not reduced by RF in said patient and/or (i) the treatment induces low disease activity or achieves substantial clinical benefit in the patient.

4. A method of treating rheumatoid arthritis in a human patient having rheumatoid factor comprising:
a) determining the titer of rheumatoid factor in a sample, preferably a serum sample, from said patient;
b) administering certolizumab pegol to said patient if the titer of rheumatoid factor is above 100 IU/ml, above 121.4 IU/ml, above 125 IU/ml, above 145.9 IU/ml, above 150 IU/ml, above 175 IU/ml, above 200 IU/ml, above 204 IU/ml, above 225 IU/ml, above 250 IU/ml, or above 275 IU/ml in serum;
wherein (i) the therapeutic effect of certolizumab pegol is not reduced by RF in said patient and/or (i) the treatment induces low disease activity or achieves substantial clinical benefit in the patient.

5. The method according to any one of claims 1-4, wherein the low disease activity is achieved 90 days, 100 days, 104 days, 120 days or 180 days after the first administration of certolizumab pegol.

6. The method according to any one of claims 1-5, wherein the low disease activity in the patient is achieved with a likelihood of 40%, 45%, 50% or 55%.

7. The method according to any one of claims 3-6 wherein certolizumab pegol is administered to the patient in step (b) at loading dose of 400 mg initially and 400 mg two weeks later followed by a maintenance dose of (i) 400 mg of every 4 weeks or (ii) 200 mg every 2 weeks.

8. The method according to any one of claims 1-7, wherein no loading dose is administered to the patient at the initiation of treatment.

9. The method according to any one of claims 1-8, wherein the rheumatoid factor is of the IgM, IgG, IgD, IgE or IgA isotype.

10. The method according to any one of claims 3-9, wherein certolizumab pegol is administered in combination with another anti-rheumatic drug.

11. The method according to claim 10, wherein the anti-rheumatic drug is methotrexate or leflunomide.

12. The method according to any one of claims 1-11, wherein the patient has previously failed to respond to treatment with a DMARD.

13. The method according to claim 12, wherein the patient has previously failed to respond to treatment with a biological DMARD having an Fc fragment.

14. The method according to claim 12, wherein the patient has previously failed to respond to treatment with a non-biological DMARD.

15. The method according to claim 13 or 14, wherein treatment failure is a primary failure.

16. The method according to claim 13 or 14, wherein treatment failure is a secondary failure.

17. The method according to any one of claims 1-16, wherein bDMARD lacking an Fc fragment is administered at regular intervals for at least 3 months.

18. The method according to any one of claims 1-17, wherein the plasma concentration of the bDMARD lacking an Fc fragment is not decreased in the patient following its administration.

19. A method of maintaining low disease activity or remission of a rheumatic disease in a human subject in need thereof, the method comprising:
administering to the subject a dose of a bDMARD lacking an Fc fragment that is effective to maintain low disease activity or remission in the subject,
wherein the subject is identified as having an elevated baseline rheumatoid factor (RF) serum level of more than 100 IU/ml.

20. The method of claim 19, wherein the subject has rheumatoid arthritis (RA).

21. The method of claim 20, wherein the subject has early RA.

22. The method of claim 20, wherein the subject had active RA for at least 6 months prior to treatment.

23. The method of any one of claims 19-22, wherein the bDMARD is certolizumab pegol.

24. The method of claim 23, wherein the certolizumab pegol is administered to the subject at a loading dose of 400 mg initially and 400 mg two weeks later followed by a maintenance dose.

25. The method of claim 23, wherein no loading dose is administered to the patient at the initiation of treatment with a maintenance dose.

26. The method of claim 25, wherein the certolizumab pegol is administered to the subject at a maintenance dose of 400 mg of every 4 weeks.

27. The method of claim 25, wherein the certolizumab pegol is administered to the subject at a maintenance dose of 200 mg every 2 weeks.

28. The method of any one of claims 19-27, wherein the bDMARD is administered to the subject for at least 12 weeks.

29. The method of any one of claims 19-28, wherein the bDMARD is administered to the subject for at least 24 weeks.

30. The method of any one of claims 19-29, wherein the bDMARD is administered to the subject for at least 1 year.

31. The method of any one of claims 19-30, wherein the bDMARD is administered to the subject for at least 2 years.

32. The method of any one of claims 19-31, wherein low disease activity or remission is achieved by 12 weeks after the first administration of the bDMARD, and maintained during the period of administration.

33. The method of any one of claims 19-32, wherein the subject is identified as having a baseline RF serum level of more than 121.4 IU/ml.

34. The method of any one of claims 19-32, wherein the subject is identified as having a baseline RF serum level of more than 125 IU/ml.

35. The method of any one of claims 19-32, wherein the subject is identified as having a baseline RF serum level of more than 145.9 IU/ml.

36. The method of any one of claims 19-32, wherein the subject is identified as having a baseline RF serum level of more than 150 IU/ml.

37. The method of any one of claims 19-32, wherein the subject is identified as having a baseline RF serum level of more than 175 IU/ml.

38. The method of any one of claims 19-32, wherein the subject is identified as having a baseline RF serum level of more than 200 IU/ml.

39. The method of any one of claims 19-32, wherein the subject is identified as having a baseline RF serum level of more than 204 IU/ml.

40. The method of any one of claims 19-32, wherein the subject is identified as having a baseline RF serum level of more than 225 IU/ml.

41. The method of any one of claims 19-32, wherein the subject is identified as having a baseline RF serum level of more than 250 IU/ml.

42. The method of any one of claims 19-32, wherein the subject is identified as having a baseline RF serum level of at least 275 IU/ml.

43. The method of any one of claims 19-43, further comprising the earlier step of determining the serum level of RF of a subject having a rheumatic disease.

44. The method of any one of claims 19-44, wherein the RF is of the IgM, IgG, IgD, IgE or IgA isotype.

45. The method of any one of claims 19-44, wherein the bDMARD is administered in combination with another anti-rheumatic drug.

46. The method according to claim 45, wherein the anti-rheumatic drug is methotrexate or leflunomide.
